(19) United States
(12) Patent Application Publication
(10) Pub. No.: US 2023/0233672 A1
(43) Pub. Date: Jul. 27, 2023

(54) METHODS OF MANUFACTURE OF NUT FLOURS AND FORMULATIONS FOR ORAL IMMUNOTHERAPY

(71) Applicant: SOCIÉTÉ DES PRODUITS NESTLÉ S.A., Vevey (CH)

(72) Inventors: Reyna Joy Simon, Los Gatos, CA (US); Nicholas William BIRRINGER, Brisbane, CA (US)

(21) Appl. No.: 18/121,826

(22) Filed: Mar. 15, 2023

Related U.S. Application Data
(62) Division of application No. 15/870,477, filed on Jan. 12, 2018.
(60) Provisional application No. 62/446,147, filed on Jan. 13, 2017.

(51) Int. Cl. A61K 39/35; A61P 37/08; A61K 9/48; A23L 25/00; A61K 39/00

(52) U.S. Cl. CPC A61K 39/35; A23L 25/30; A61K 9/48; A61P 37/08; A23V 2002/00; A61K 2039/542; A61K 2039/545

(57) ABSTRACT

Methods of manufacture of nut flours and/or formulations, ultra-low fat nut flours and uses for nut flour formulations. In some embodiments, methods of manufacturing ultra-low fat tree nut or peanut flour formulations for oral administration in immunotherapy of subjects affected by allergies.

### CROSS-REFERENCE TO RELATED APPLICATION

This application is a divisional of U.S. Pat. Appl. Serial No. 15/870,477 filed Jan. 12, 2018, which claims priority to U.S. Provisional Application No. 62/446,147 filed Jan. 13, 2017, which are entirely incorporated herein by reference for all purposes.

### TECHNICAL FIELD

The present technology relates generally to methods of manufacture of nut flours and/or formulations, ultra-low fat nut flours and uses for nut protein formulations. In particular, several embodiments are directed to methods of manufacturing ultra-low fat tree nut or peanut protein formulations for oral administration in immunotherapy of subjects affected by allergies.

### BACKGROUND

Allergies, or a body’s immunological reaction to a foreign substance (e.g., insects, foods, latex, drugs, etc.), can affect both humans and animals. In the case of food allergies, such a foreign substance can include allergenic epitopes from proteins in the food, such as protein fragments or amino acid structures including both linear epitopes and conformational epitopes that are offending to the subject’s immune system. The severity of allergic reactions can vary between individuals and can range from between mild irritation to anaphylaxis, which can be so severe as to be life threatening.

Peanut and tree nut allergies are relatively common in western societies, with the estimated prevalence of peanut allergies being approximately 1.5% -2% in western countries and the prevalence of tree nut allergy ranging from ~1% in the U.S. to 1.4% in the EU. Allergic cross-reactivity may exist between peanuts and a number of tree nuts. In the U.S. approximately 30% of peanut allergic individuals are allergic to tree nuts and vice versa. The Food Allergy and Anaphylaxis Network’s peanut and tree nut registry found significant numbers of respondents reporting allergies to various tree nuts, including: walnuts (34%); cashews (20%); almonds (15%); pecans (9%); pistachios (7%); and other nuts (<5% each).

Treating patients for peanut or tree nut allergies using oral immunotherapy is subject to accurate dosing, purity and correct protein characterizing of administered formulations. Peanut or tree nut flours, which provide the basis for administered formulations, can however, go rancid due to the fats and oils remaining in the nut flours. For example, it is known that reducing the fat content of peanut and tree nut flours increases the stability of the respective flours; however, it has been previously disclosed that lowering the fat content of nut flour yielded a hypoallergenic product (see U.S. Pat. Publication No. 2012/0164306).

Food allergies are caused, in most cases, by a reaction to proteins in the food. In the early years of life, the immune system is still developing and may fail to develop tolerance to dietary antigens (this may also be described as insufficient induction of oral tolerance). The result is that the baby or child or young animal mounts an exaggerated immune response to the dietary protein and develops an allergic response to it. The most common food allergies in children are milk, eggs, peanuts, and tree nuts. Currently there are no effective treatments available for food allergy. Avoiding the offending allergen has been the only accepted strategy to manage food allergy. However, strict avoidance diets can be complicated due to difficulty in interpreting labels and by the presence of undeclared or hidden allergens in commercially prepared foods.

Specific immunotherapy for food allergy, including peanut and tree nut allergies, in the forms of oral immunotherapy (OIT) and sublingual immunotherapy (SLIT) has been studied in recent years and has demonstrated encouraging safety and efficacy results in early clinical trials, including beneficial immunologic changes. OIT has shown evidence for inducing desensitization in most subjects with immunologic changes over time indicating progression toward clinical tolerance (Skripak et. al., J. Allergy Clin Immunol. 122(6): 1154-1160, 2008; Keet et. al., J. Allergy Clin Immunol. 129(2): 448-455, 2012).

In view of the prevalence of peanut and tree nut allergies, there is a need for stable, therapeutic compounds and allergenic compositions to use in treatments to reduce the severity of individuals’ reactions to these common food items.

### SUMMARY OF THE INVENTION

Described herein are nut flour compositions, including tree nut flour compositions and peanut flour compositions. Also described are methods of making such nut flour compositions and method of using such nut flour compositions, including for methods of treatment of a nut allergy.

In some embodiments, there is a tree nut flour composition comprising defatted tree nut flour, wherein at least 50% of the defatted tree nut flour by weight passes through a 250 µm sieve. In some embodiments, the tree nut flour further comprises a carrier material. In some embodiments, the carrier material comprises one or more diluents, glidants, or lubricants.

In some embodiments, the defatted tree nut flour has an oil content of less than about 12% by weight. In some embodiments, the defatted tree nut flour has an oil content of less than about 6% by weight.

In some embodiments, the tree nut is walnut, almond, pecan, cashew, hazelnut, pine nut, brazil nut, or pistachio.

In some embodiments, approximately all of the defatted tree nut flour passes through a 1 mm sieve. In some embodiments, approximately all of the defatted tree nut flour passes through a 250 µm sieve. In some embodiments, approximately all of the defatted tree nut flour passes through a 149 µm sieve. In some embodiments, approximately all of the defatted tree nut flour passes through a 74 µm sieve.

In some embodiments, the defatted tree nut flour is produced according to a method comprising contacting a tree nut material with a supercritical fluid; and milling the tree nut material to form the defatted tree nut flour. In some embodiments, the supercritical fluid is supercritical carbon dioxide.

In some embodiments, the tree nut flour composition is combined with a food stuff.

In some embodiments, there is a method of treating nut allergy in a subject, comprising orally administering to the subject an effective amount of the tree nut flour composition described above.

In some embodiments, there is a dosage form for oral immunotherapy, comprising the tree nut flour composition described above. In some embodiments, the dosage form comprises a measured amount of the tree nut flour composition. In some embodiments, the dosage form comprises about 0.1 mg to about 2000 mg of tree nut flour. In some embodiments, the measured dose comprises about 0.5 mg to about 1000 mg nut protein. In some embodiments, the tree nut flour composition is enclosed in a package. In some embodiments, the package identifies an amount of nut protein or an amount of nut flour contained within the dosage form or package. In some embodiments, the tree nut flour composition is encapsulated in a capsule.

In some embodiments, there is a dosage form for oral immunotherapy, comprising a measured amount of a defatted nut flour, wherein the defatted nut flour is produced according to a method comprising contacting a nut material with a supercritical fluid, thereby reducing the oil content of the nut material to form a defatted nut flour; and measuring a dose of the defatted nut flour. In some embodiments, the measured dose comprises about 0.1 mg to about 2000 mg nut protein. In some embodiments, the measured dose comprises about 0.5 mg to about 1000 mg nut protein. In some embodiments, the method of producing the defatted nut flour further comprises milling the defatted nut flour. In some embodiments, the method of producing the defatted nut flour further comprises pressing or milling the nut material prior to contacting the nut material with the supercritical fluid. In some embodiments, the defatted nut flour is combined with a carrier material. In some embodiments, the carrier material comprises one or more diluents, glidants, or lubricants. In some embodiments, the defatted nut flour is enclosed in a package. In some embodiments, the package identifies an amount of nut protein or an amount of nut flour contained within the dosage form or package. In some embodiments, the defatted nut flour is encapsulated in a capsule.

In some embodiments of the dosage form, at least 50% of the defatted nut flour by weight passes through a 250 µm sieve. In some embodiments, approximately all of the defatted nut flour passes through a 1 mm sieve. In some embodiments, approximately all of the defatted tree nut flour passes through a 250 µm sieve. In some embodiments, approximately all of the defatted tree nut flour passes through a 149 µm sieve. In some embodiments, approximately all of the defatted tree nut flour passes through a 74 µm sieve.

In some embodiments of the dosage form, the defatted nut flour has an oil content of less than about 12%. In some embodiments, the defatted nut flour has an oil content of less than about 6%.

In some embodiments of the dosage form, the defatted nut flour is a peanut flour. In some embodiments, the defatted nut flour is a tree nut flour. In some embodiments, the tree nut is walnut, almond, pecan, cashew, hazelnut, pine nut, brazil nut, or pistachio.

In some embodiments of the dosage form, the supercritical fluid is supercritical carbon dioxide.

In some embodiments, there is a kit comprising a dosage form as described above and instructions for use. In some embodiments, the instructions for use comprise instructions for combining the defatted nut flour with a food stuff. In some embodiments, the instructions for use comprise instructions for daily administration of the dosage form.

In some embodiments, there is a method of manufacturing an ultra-low fat nut material comprising contacting a nut material having an initial oil content with a supercritical fluid to provide a defatted nut material having a reduced oil content; and milling the defatted nut material.

In some embodiments, there is a method of manufacturing an ultra-low fat nut material comprising contacting a nut material having an initial oil content with a supercritical fluid to provide a defatted nut material having a reduced oil content; and measuring a dose of the defatted nut material for oral immunotherapy. In some embodiments, the measured dose of the defatted nut material comprises about 0.1 mg to about 1000 mg nut protein.

In some embodiments, there is a method of manufacturing an ultra-low fat nut material comprising contacting a nut material having an initial oil content with a supercritical fluid to provide a defatted nut material having a reduced oil content; and packaging the defatted nut material in a package. In some embodiments, the package has a volume of less than 5 mL. In some embodiments, the package is a capsule.

In some embodiments of any of the methods described above, the method comprises combining the defatted nut material with a carrier material. In some embodiments, the carrier material comprises one or more diluents, glidants, or lubricants.

In some embodiments of any of the methods described above, the defatted nut material has an oil content between 0.1% and 12%.

In some embodiments of any of the methods described above, the supercritical fluid is carbon dioxide.

In some embodiments of any of the methods described above, the nut material is a peanut material. In some embodiments, the nut material is a tree nut material. In some embodiments, the tree nut material is a walnut material, a cashew material, a hazelnut material, an almond material, a pistachio material, a pine nut material, a brazil nut material, or a pecan material.

In some embodiments of any of the methods described above, the method comprises pressing or milling the nut material prior to contacting the nut material with the supercritical fluid.

In some embodiments of any of the methods described above, the method comprises characterizing one or more nut protein allergens in the defatted nut material. In some embodiments, the one or more nut protein allergens are characterized using an enzyme linked immunosorbent assay (ELISA), reversed-phase high performance liquid chromatography (HPLC), size-exclusion chromatography (SEC), mass spectrometry, liquid chromatography-mass spectrometry (LC-MS), liquid chromatography-mass spectrometry/mass spectrometry (LC-MS/MS), or an immunoblot.

In some embodiments of any of the methods described above, the method comprises separating larger particles in the nut flour from smaller nut particles in the nut flour, and retaining the smaller nut particles. In some embodiments, the larger particles are separated from the smaller nut particles using one or more sieves.

In some embodiments of any of the methods described above, contacting the nut material with the supercritical fluid comprises flowing the supercritical fluid through the nut material.

In some embodiments, there is a method of treating a tree nut allergy in a subject, comprising orally administering to the subject an effective amount of a pharmaceutical composition comprising defatted tree nut flour, wherein the effective amount of the pharmaceutical composition comprises about 0.1 mg to about 2000 mg tree nut protein. In some embodiments, the effective amount of the pharmaceutical composition comprises about 0.5 mg to about 1000 mg tree nut protein.

In some embodiments of any of the methods described above, at least 50% of the defatted tree nut flour by weight passes through a 250 µm sieve. In some embodiments, approximately all of the defatted tree nut flour passes through a 1 mm sieve. In some embodiments, approximately all of the defatted tree nut flour passes through a 250 µm sieve. In some embodiments, approximately all of the defatted tree nut flour passes through a 149 µm sieve. In some embodiments, approximately all of the defatted tree nut flour passes through a 74 µm sieve.

In some embodiments of any of the methods described above, the defatted tree nut flour has an oil content of less than about 12% by weight. In some embodiments, the defatted tree nut flour has an oil content of less than about 6% by weight.

In some embodiments of any of the methods described above, the tree nut is walnut, almond, pecan, cashew, hazelnut, pine nut, brazil nut, or pistachio.

In some embodiments, there is a method of treating a nut allergy in a subject, comprising orally administering to the subject an effective amount of a pharmaceutical composition comprising defatted nut flour, wherein the defatted nut flour is produced according to a method comprising contacting a nut material with a supercritical fluid, thereby reducing the oil content of the nut material to form the defatted nut flour. In some embodiments, the effective amount of the pharmaceutical composition comprises about 0.1 mg to about 2000 mg nut protein. In some embodiments, the effective amount of the pharmaceutical composition comprises about 0.5 mg to about 1000 mg nut protein.

In some embodiments of any of the methods described above, the method of producing the defatted nut flour further comprises milling the defatted nut flour. In some embodiments, the method of producing the defatted nut flour further comprises pressing or milling the nut material prior to contacting the nut material with the supercritical fluid.

In some embodiments of any of the methods described above, at least 50% of the defatted nut flour by weight passes through a 250 µm sieve. In some embodiments, approximately all of the defatted nut flour passes through a 1 mm sieve. In some embodiments, approximately all of the defatted nut flour passes through a 250 µm sieve. In some embodiments, approximately all of the defatted nut flour passes through a 149 µm sieve. In some embodiments, approximately all of the defatted tree nut flour passes through a 74 µm sieve.

In some embodiments of any of the methods described above, the defatted nut flour has an oil content of less than about 12% by weight. In some embodiments, the defatted nut flour has an oil content of less than about 6% by weight.

In some embodiments of any of the methods described above, the defatted nut flour is a peanut flour. In some embodiments, the defatted nut flour is a tree nut flour. In some embodiments, the tree nut is walnut, almond, pecan, cashew, hazelnut, pine nut, brazil nut, or pistachio.

In some embodiments of any of the methods described above, the supercritical fluid is supercritical carbon dioxide. In some embodiments, the pharmaceutical composition comprises a carrier material. In some embodiments, the carrier material comprises one or more diluents, glidants, or lubricants.

In some embodiments of any of the methods described above, the pharmaceutical composition is administered daily. In some embodiments, the same effective amount of the pharmaceutical composition is administered daily for at least one week. In some embodiments, the effective amount of the pharmaceutical composition is periodically increased. In some embodiments, the effective amount of the pharmaceutical composition is adjusted to a different effective amount of the pharmaceutical composition after a period of at least one week, wherein the different effective amount of the pharmaceutical composition comprises about 0.1 mg to about 2000 mg tree nut protein. In some embodiments, the effective amount of the pharmaceutical composition is adjusted to a different effective amount of the pharmaceutical composition after a period of at least one week, wherein the different effective amount of the pharmaceutical composition comprises about 0.5 mg to about 1000 mg tree nut protein. In some embodiments, the pharmaceutical composition is administered daily for at least one month.

In some embodiments of any of the methods described above, the subject is a human.

### BRIEF DESCRIPTION OF THE DRAWINGS

FIG. 1 shows the results of SDS-PAGE separated proteins from supercritical CO₂ (sCO₂) extracted peanut material (SEP) in accordance with embodiments disclosed hereof.

FIG. 2 shows immunoblot analysis of SDS-PAGE separated proteins from supercritical CO₂ extracted peanut material in accordance with embodiments disclosed hereof.

FIG. 3 shows immunoblot analysis of SDS-PAGE separated proteins from supercritical CO₂ extracted peanut material using pooled patient antisera in accordance with embodiments disclosed hereof.

FIG. 4 is a chromatogram overlay of RP-HPLC separated proteins from supercritical CO₂ extracted peanut material (SEP) and a profile from a reference peanut flour in accordance with embodiments disclosed hereof.

FIG. 5 is a chromatogram overlay of RP-HPLC separated proteins from GPC peanut flour and a peanut flour reference standard in accordance with embodiments disclosed hereof.

FIGS. 6A-6C show detection of Ara h 1 (FIG. 6A), Ara h 2 (FIG. 6B), and Ara h 6 (FIG. 6C) in supercritical CO₂ extracted peanut (SEP) material (circles) and a reference standard peanut flour (squares) by Enzyme Linked Immunosorbent Assays (ELISA) in accordance with embodiments disclosed hereof.

FIG. 7 is a graph illustrating total protein extracted from supercritical CO₂ extracted walnut materials and pressed walnut materials in accordance with embodiments disclosed hereof.

FIG. 8 shows the result of SDS-PAGE separated proteins from petroleum ether extracted walnut material reference sample, supercritical CO₂ extracted walnut materials, and hexane extracted walnut materials in accordance with embodiments disclosed hereof.

FIG. 9 shows immunoblot analysis of SDS-PAGE separated proteins from petroleum ether extracted walnut material reference sample, supercritical CO₂ extracted walnut materials, and hexane extracted walnut materials in accordance with embodiments disclosed hereof.

FIG. 10 shows particle size distribution for supercritical CO₂ extracted peanut (SEP) material in accordance with embodiments disclosed hereof.

FIG. 11 shows particle size distribution for multiple lots of commercially available peanut flour. Left panel shows the percent of peanut flour retained (by weight) for each screen mesh size for each lot, with each lot displayed from left to right in the same order as identified from top to bottom in the key, for each mesh size. Right panel shows the cumulative percent retained.

FIG. 12A shows particle size distribution for dry roasted walnut flour obtained in accordance with embodiments disclosed hereof.

FIG. 12B shows particle size distribution for raw walnut flour obtained in accordance with embodiments disclosed hereof.

FIG. 13 shows particle size distribution for raw pecan flour obtained in accordance with the embodiments disclosed hereof.

FIG. 14A shows an HPLC trace of proteins extracted from defatted walnut flour prepared by extracting oil from pasteurized walnut material using supercritical carbon dioxide. Arrows indicate specific protein antigen peaks.

FIG. 14B shows an HPLC trace of proteins extracted from defatted walnut flour prepared by extracting oil from pasteurized walnut material using a screw oil expeller followed by extraction using supercritical carbon dioxide. Arrows indicate specific protein antigen peaks.

FIG. 14C shows an HPLC trace of proteins extracted from a commercially available defatted walnut flour (Bio Planete, Lommatzsch, Germany). Arrows indicate specific protein antigen peaks.

FIG. 15A shows an immunoblot of walnut protein extracts stained with pooled sera obtained from individuals with a diagnosed walnut allergy. The samples in the various lanes as follows: (1) steam-pasteurized walnut material defatted using supercritical carbon dioxide at about 45° C.; (2) steam-pasteurized walnut material defatted using supercritical carbon dioxide at about 75° C.; (3) commercially available walnut flour (Lot No. 08340148; Bio Planete, Lommatzsch, Germany); (4) commercially available walnut flour (Lot No. 07210081; Bio Planete, Lommatzsch, Germany); and (5) unpasteurized walnut material defatted using supercritical carbon dioxide at about 45° C. J2 indicates isolated Jug r 2, and J1 indicates isolated Jug r 1.

FIG. 15B shows an immunoblot of walnut protein extracts stained with an HC121 antibody, which recognizes both Jug r 1 and Jug r 2. The samples in the various lanes as follows: (1) steam-pasteurized walnut material defatted using supercritical carbon dioxide at about 45° C.; (2) steam-pasteurized walnut material defatted using supercritical carbon dioxide at about 75° C.; (3) commercially available walnut flour (Lot No. 08340148; Bio Planete, Lommatzsch, Germany); (4) commercially available walnut flour (Lot No. 07210081; Bio Planete, Lommatzsch, Germany); and (5) unpasteurized walnut material defatted using supercritical carbon dioxide at about 45° C. J2 indicates isolated Jug r 2, and J1 indicates isolated Jug r 1.

### DETAILED DESCRIPTION

### I. Overview

Various aspects of the present technology provide methods of manufacturing and using low fat and/or ultra-low fat pharmaceutical preparations of various nut flours extracted with super critical fluid, such as supercritical CO₂, extraction methods that retain their allergenicity and are, therefore, useful in oral immunotherapy regimes. The ultra-low fat content of such preparations renders such products more stable and less susceptible to rancidity issues than nut flours having higher fat contents. Additionally, defatted nut flours are more easily milled into smaller particle size, which increases fluidity of the nut flour and facilitates accurate measurements of therapeutic doses. It is important that doses for oral immunotherapy be accurately measured to avoid accidental inducement of an anaphylactic reaction.

Furthermore, in certain embodiments including but not limited to those relating to walnut flour, the resulting preparations have the unexpected property of having more easily extractable proteins than nut flours defatted through other methods. Surprisingly, super-critical CO₂ purification methods also render the preparations less aromatic, reducing the smell and taste of the nuts in the final product, as well as lighter in color. As many allergic patients report an aversion to the smell and taste of foods to which they are allergic, removal of the aromatic components in an oral immunotherapy therapeutic formulation is expected to improve patient compliance with the immunotherapy regime. Nevertheless, despite the harsh conditions of supercritical CO₂ defatting of nut flour, the nut flours retain their antigenic properties.

Additional aspects of the present technology provide formulations comprising ultra-low fat nut protein powder that may be formulated into a pharmaceutical composition. These presently disclosed formulations, when administered to a patient according to a treatment regimen, can provide oral immunotherapy (OIT) for subjects that are allergic to peanut and/or tree nut products. Following treatment, subjects administered an oral food challenge (OFC) may be partially or fully desensitized to peanut protein and/or tree nut protein(s) in accordance with aspects of the present technology.

Specific details of several embodiments of the technology are described below in the Detailed Description and the Examples. Although many of the embodiments are described below with respect to methods of manufacture of ultra-low fat nut protein powder and compositions (i.e., formulations) comprising ultra-low fat nut protein powder for oral immunotherapy and/or for use in clinical trials for oral immunotherapy of peanut protein and/or tree nut protein(s), other applications and other embodiments in addition to those described herein are within the scope of the technology. Additionally, several other embodiments of the technology can have different components or procedures than those described herein. A person of ordinary skill in the art, therefore, will accordingly understand that the technology can have other embodiments with additional components, or the technology can have other embodiments without several of the aspects shown and described below.

All references, including publications, patent applications, and patents, cited herein are hereby incorporated by reference to the same extent as if each reference were individually and specifically indicated to be incorporated by reference and were set forth in its entirety herein.

### II. Definitions

Unless defined otherwise, all technical and scientific terms used herein have the same meaning as is commonly understood by one of skill in the art to which the present technology described herein belong. All patents and publications referred to herein are incorporated by reference.

The term “animal”, as used herein, refers to humans as well as non-human animals, including, for example, mammals, birds, reptiles, amphibians, and fish. In some embodiments, the non-human animal is a mammal (e.g., a rodent, a mouse, a rat, a rabbit, a monkey, a dog, a cat, a primate, or a pig). In further embodiments, an animal may be a transgenic animal.

As used herein, “nut” or “nut protein”, without further modification as to source, may refer to either a peanut or a tree nut.

The term “antigen”, as used herein, refers to a molecule that elicits production of an antibody response (i.e., a humoral response) and/or an antigen-specific reaction with T-cells (i.e., a cellular response) in an animal.

The term “allergen”, as used herein, refers to a subset of antigens which elicit the production of IgE in addition to other isotypes of antibodies. The terms “allergen”, “natural allergen”, and “wild-type allergen” may be used interchangeably. Some examples of allergens for the purpose of the present technology are protein allergens.

The phrase “allergic reaction”, as used herein, relates to an immune response that is IgE mediated with clinical symptoms primarily involving the cutaneous (e.g., uticaria, angioedema, pruritus), respiratory (e.g., wheezing, coughing, laryngeal edema, rhinorrhea, watery/itching eyes), gastrointestinal (e.g., vomiting, abdominal pain, diarrhea), and cardiovascular (i.e., if a systemic reaction occurs) systems. For the purposes of the present technology, an asthmatic reaction is considered to be a form of allergic reaction.

The phrase “anaphylactic allergen”, as used herein, refers to a subset of allergens that are recognized to present a risk of anaphylactic reaction in allergic individuals when encountered in its natural state, under natural conditions. For example, for the purposes of the present technology, pollen allergens, mite allergens, allergens in animal dander or excretions (e.g., saliva, urine), and fungi allergens are not considered to be anaphylactic allergens. On the other hand, food allergens, insect allergens, and rubber allergens (e.g., from latex) are generally considered to be anaphylactic allergens. Food allergens, in particular, are anaphylactic allergens for use in the practice of the present technology. In particular, nut allergens (e.g., from peanut, walnut, almond, pecan, cashew, hazelnut, pistachio, pine nut, brazil nut, etc.), egg/dairy allergens (e.g., from egg, milk, etc.), seed allergens (e.g., from sesame, poppy, mustard, etc.), soybean, wheat, and fish/shellfish allergens (e.g., from shrimp, crab, lobster, clams, mussels, oysters, scallops, crayfish, etc.) are anaphylactic food allergens according to the present technology. Particularly interesting anaphylactic allergens are those to which reactions are commonly so severe as to create a risk of death.

The phrase “anaphylaxis” or “anaphylactic reaction”, as used herein, refers to a subset of allergic reactions characterized by mast cell degranulation secondary to cross-linking of the high-affinity IgE receptor on mast cells and basophils induced by an anaphylactic allergen with subsequent mediator release and the production of severe systemic pathological responses in target organs, e.g., airway, skin digestive tract, and cardiovascular system. As is known in the art, the severity of an anaphylactic reaction may be monitored, for example, by assaying cutaneous reactions, puffiness around the eyes and mouth, vomiting, and/or diarrhea, followed by respiratory reactions such as wheezing and labored respiration. The most severe anaphylactic reactions can result in loss of consciousness and/or death.

The phrase “antigen presenting cell” or “APC”, as used herein, refers to cells which process and present antigens to T-cells to elicit an antigen-specific response, e.g., macrophages and dendritic cells.

When two entities are “associated with” one another as described herein, they are linked by a direct or indirect covalent or non-covalent interaction. Preferably, the association is covalent. Desirable non-covalent interactions include, for example, hydrogen bonding, van der Walls interactions, hydrophobic interactions, magnetic interactions, etc.

The phrase “decreased anaphylactic reaction”, as used herein, relates to a decrease in clinical symptoms following treatment of symptoms associated with exposure to an anaphylactic allergen, which can involve exposure via cutaneous, respiratory, gastrointestinal, and mucosal (e.g., ocular, nasal, and aural) surfaces or a subcutaneous injection (e.g., via a bee sting).

Desensitization″ or “desensitize” refers to the ability of a patient to consume small to large amounts of the allergic food source without demonstrating an allergic reaction. Desensitization differs from “tolerance” in that it requires chronic treatment with the food source to maintain the “allergic-free” state. Whereas in the “tolerance” state, treatment is no longer required.

The term “epitope”, as used herein, refers to a binding site including an amino acid motif of between approximately six and fifteen amino acids which can be bound by an immunoglobulin (e.g., IgE, IgG, etc.) or recognized by a T-cell receptor when presented by an APC in conjunction with the major histocompatibility complex (MHC). A linear epitope is one where the amino acids are recognized in the context of a simple linear sequence. A conformational epitope is one where the amino acids are recognized in the context of a particular three dimensional structure.

An allergen “fragment” according to the present technology is any part or portion of the allergen that is smaller than the intact natural allergen. In certain embodiments of the present technology, the allergen is a protein and the fragment is a peptide.

The phrase “immunodominant epitope”, as used herein, refers to an epitope which is bound by antibody in a large percentage of the sensitized population or where the titer of the antibody is high, relative to the percentage or titer of antibody reaction to other epitopes present in the same antigen. In one embodiment, an immunodominant epitope is bound by antibody in more than 50% of the sensitive population and, in further examples, more than 60%, 70%, 80%, 90%, 95%, or 99%.

“Isolated” (used interchangeably with “substantially pure”) when applied to polypeptides means a polypeptide or a portion thereof, which has been separated from other proteins with which it naturally occurs. Typically, the polypeptide is also substantially (i.e., from at least about 70% to about 99%) separated from substances such as antibodies or gel matrices (polyacrylamide) which are used to purify it.

“Absorption” typically refers to the process of movement of a delivered substance (e.g., nut protein allergen(s)) from the gastrointestinal tract into a blood vessel.

“Bioavailability” refers to the percentage of the weight of nut protein allergen(s) dosed that is delivered into the general circulation of the animal or human being studied. The total exposure (AUC(0-00)) of a drug when administered intravenously is usually defined as 100% Bioavailable (F%). “Oral bioavailability” refers to the extent to which nut protein allergen(s) are absorbed into the general circulation when the pharmaceutical composition is taken orally as compared to intravenous injection.

“Blood plasma concentration” refers to the concentration of a nut protein allergen(s) in the plasma component of blood of a subject. It is understood that the plasma concentration of nut protein allergen(s) may vary significantly between subjects, due to variability with respect to metabolism and/or possible interactions with other therapeutic agents. In accordance with one aspect of the present invention, the blood plasma concentration of nut protein allergen(s) may vary from subject to subject. Likewise, values such as maximum plasma concentration (Cmax) or time to reach maximum plasma concentration (Tmax), or total area under the plasma concentration time curve (AUC(0-00)) may vary from subject to subject. Due to this variability, the amount necessary to constitute “a therapeutically effective amount” of nut protein allergen(s) may vary from subject to subject.

A “measurable serum concentration” or “measurable plasma concentration” describes the blood serum or blood plasma concentration, typically measured in mg, µg, or ng of therapeutic agent per mL, dL, or L of blood serum, absorbed into the bloodstream after administration. As used herein, measurable plasma concentrations are typically measured in ng/mL or µg/mL.

“Oral food challenge” refers to a highly accurate diagnostic test for food allergy. During the food challenge, the allergist feeds the patient the suspect food in measured doses, starting with very small amounts that are unlikely to trigger symptoms. Following each dose, the patient is observed for a period of time for any signs of a reaction. If there are no symptoms, the patient gradually receives increasingly larger doses. If any signs of a reaction are evident, the food challenge is stopped and the patient is characterized as failing the food challenge and is allergic to the food at the sensitivity level determined by the amount of food triggering the allergic response.

“Oral immunotherapy” refers to an orally-administered medical treatment for patients suffering from allergies, involving administering increasing doses of allergens to the patients in order to desensitize or provide tolerance to a patient for that allergen.

“Pharmacodynamics” refers to the factors which determine the biologic response observed relative to the concentration of drug at a site of action.

“Pharmacokinetics” refers to the factors which determine the attainment and maintenance of the appropriate concentration of drug at a site of action.

“Carrier materials” include any commonly used excipients in pharmaceutics and should be selected on the basis of compatibility with nut protein allergen(s) and the release profile properties of the desired dosage form. Exemplary carrier materials include, e.g., binders, suspending agents, disintegration agents, filling agents, surfactants, solubilizers, stabilizers, lubricants, wetting agents, diluents, and the like.

“Pharmaceutically compatible carrier materials” may comprise, but are not limited to, acacia, gelatin, colloidal silicon dioxide, calcium glycerophosphate, calcium lactate, maltodextrin, glycerine, magnesium silicate, polyvinylpyrrollidone (PVP), cholesterol, cholesterol esters, sodium caseinate, soy lecithin, taurocholic acid, phosphotidylcholine, sodium chloride, tricalcium phosphate, dipotassium phosphate, cellulose and cellulose conjugates, sugars sodium stearoyl lactylate, carrageenan, monoglyceride, diglyceride, pregelatinized starch, and the like. See, e.g., Remington: The Science and Practice of Pharmacy, Nineteenth Ed (Easton, Pa.: Mack Publishing Company, 1995); Hoover, John E., Remington’s Pharmaceutical Sciences, Mack Publishing Co., Easton, Pennsylvania 1975; Liberman, H.A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

“Plasticizers” are compounds which may be used to soften the microencapsulation material or film coatings to make them less brittle. Suitable plasticizers include, e.g., polyethylene glycols such as PEG 300, PEG 400, PEG 600, PEG 1450, PEG 3350, and PEG 800, stearic acid, propylene glycol, oleic acid, triethyl cellulose and triacetin. In some embodiments, plasticizers can also function as dispersing agents or wetting agents.

“Solubilizers” include compounds such as triacetin, triethylcitrate, ethyl oleate, ethyl caprylate, sodium lauryl sulfate, sodium doccusate, vitamin E TPGS, dimethylacetamide, N-methylpyrrolidone, N-hydroxyethylpyrrolidone, polyvinylpyrrolidone, hydroxypropylmethyl cellulose, hydroxypropyl cyclodextrins, ethanol, n-butanol, isopropyl alcohol, cholesterol, bile salts, polyethylene glycol 200-600, glycofurol, transcutol, propylene glycol, dimethyl isosorbide and combinations thereof.

The total peanut and/or tree nut protein, in the ultra-low fat nut flours provided herein may be considered “stable” if its concentration is ±10% the original concentration of such protein(s) in the nut protein formulation immediately after manufacture. In another embodiment, the nut protein formulations provided herein may be considered “stable” if the nut protein formulation does not have significant changes in moisture content, appearance and odor for over three months of storage (e.g., storage at 40° C./75% relative humidity).

The compositions described herein can be formulated for administration to a subject via any conventional means including, but not limited to, oral administration routes. As used herein, the term “subject” is used to mean an animal, preferably a mammal, including a human or non-human. The flours and formulations incorporating such flours are for prevention and treatment of symptoms associated with exposure to limited amounts of nut allergen in children and adults. In one embodiment, a subject is from about 1 to about 26 years of age. In alternative embodiments, a subject may be older than 26 years of age (e.g., between 26 and 50 years of age).

A “therapeutically effective amount” or “effective amount” is that amount of nut protein allergen(s) to achieve a pharmacological effect. The term “therapeutically effective amount” includes, for example, a prophylactically effective amount. An “effective amount” of nut protein allergen(s) is an amount effective to achieve a desired pharmacologic effect or therapeutic improvement without undue adverse side effects. The effective amount of a nut protein allergen(s) will be selected by those skilled in the art depending on the particular subject and the disease level. It is understood that “an effect amount” or “a therapeutically effective amount” can vary from subject to subject, due to variation in metabolism, age, weight, general condition of the subject, the condition being treated, the severity of the condition being treated, and the judgment of the prescribing physician.

“Tolerance” to an allergen refers to the relatively long-lasting effects of immunotherapy, presumably due to effects on T cell responsiveness, that persist even after the treatment is discontinued (although tolerance may not always be permanent).

“Treat” or “treatment” as used in the context of an allergy-related disorder refers to any treatment of a disorder or disease related to allergy, such as preventing the disorder or disease from occurring in a subject which may be predisposed to the disorder or disease, but has not yet been diagnosed as having the disorder or disease; inhibiting the disorder or disease, e.g., arresting the development of the disorder or disease, relieving the disorder or disease, causing regression of the disorder or disease, relieving a condition caused by the disease or disorder, or stopping the symptoms of the disease or disorder.

As used herein, the terms “comprising,” “including,” and “such as” are used in their open, non-limiting sense.

The term “about” is used synonymously with the term “approximately.” As one of ordinary skill in the art would understand, the exact boundary of “about” will depend on the component of the composition. Illustratively, the use of the term “about” indicates that values slightly outside the cited values, i.e., plus or minus 0.1% to 10%, which are also effective and safe. In another embodiment, the use of the term “about” indicates that values slightly outside the cited values, i.e., plus or minus 0.1% to 5%, which are also effective and safe. In another embodiment, the use of the term “about” indicates that values slightly outside the cited values, i.e., plus or minus 0.1% to 2%, which are also effective and safe.

### III. Methods of Manufacture for Ultra-Low Fat Nut Flour Compositions

Provided herein are methods of manufacture for ultra-low fat peanut flour and associated compositions and/or ultra-low fat tree nut flour and associated compositions for use in oral immunotherapy.

In some embodiments, a method of manufacturing an allergenic ultra-low fat nut material (i.e., a defatted nut flour) includes contacting a nut material with a supercritical fluid to provide the defatted nut material. The defatted nut material has a reduced oil content compared to the initial oil content of the original nut material. In some embodiments, the method further comprises milling the defatted nut material, measuring a dose of the defatted nut material for oral immunotherapy, and/or packaging the defatted nut material in a package, such as a capsule.

Exemplary supercritical fluids that can be used in the invention described herein include supercritical carbon dioxide (CO₂), supercritical ethane, supercritical propane and supercritical dimethyl ether.

The supercritical fluid is heated and pressurized to a temperature and pressure above the supercritical point of the fluid. In some embodiments, the fluid is pressurized to about 7.4 MPa or higher, about 10 MPa or higher, about 20 MPa or higher, about 30 MPa or higher, about 40 MPa or higher, about 50 MPa or higher, about 60 MPa or higher, or about 70 MPa or higher. In some embodiments, the fluid is pressurized to about 80 MPa or lower, about 70 MPa or lower, about 60 MPa or lower, about 50 MPa or lower, about 40 MPa or lower, about 30 MPa or lower, or about 20 MPa or lower. In some embodiments, the temperature of the supercritical fluid is about 31° C. or warmer, about 35° C. or warmer, about 40° C. or warmer, about 45° C. or warmer, about 50° C. or warmer, or about 55° C. or warmer. In some embodiments, the supercritical fluid is about 80° C. or cooler, about 70° C. or cooler, about 60° C. or cooler, about 55° C. or cooler, about 50° C. or cooler, about 45° C. or cooler, about 40° C. or cooler, or about 35° C. or cooler.

In certain embodiments, the supercritical fluid continuously flows through the nut material to remove the fat. As the supercritical fluid flows through the nut material, fat dissolves in the supercritical fluid and is carried away. In some embodiments, the supercritical fluid flows through the nut material for about 15 minutes or more (such as about 30 minutes or more, about 1 hour or more, about 2 hours or more, about 4 hours or more, or about 6 hours or more, or about 8 hours or more). In some embodiments, the supercritical fluid flows through the nut material for about 15 minutes to about 12 hours (such as about 15 minutes to about 30 minutes, about 30 minutes to about 1 hour, about 1 hour to about 2 hours, about 2 hours to about 4 hours, about 4 hours to about 6 hours, about 6 hours to about 8 hours, or about 8 hours to about 12 hours). The flow rate of the supercritical fluid can be set depending on the desired efficiency of extraction.

In some embodiments, fat is extracted from the nut material in a batch process or a semi-batch process. For example, in some embodiments, the nut material is contacted with the supercritical fluid and held for a period of time before the supercritical fluid is removed from the nut material. The nut material can be contacted with the supercritical fluid by flowing the supercritical fluid to the nut material. The nut material can the immersed in the supercritical fluid while the fats in the nut material are extracted into the supercritical fluid. The supercritical fluid can be removed from the nut material, for example, by replacing the supercritical fluid containing the extracted fat with fresh supercritical fluid. In some embodiments, the nut material is immersed in the supercritical fluid for about 15 minutes or more (such as about 30 minutes or more, about 1 hour or more, about 2 hours or more, about 4 hours or more, or about 6 hours or more, or about 8 hours or more) before being removed. In some embodiments, the nut material is immersed in the supercritical fluid for about 15 minutes to about 12 hours (such as about 15 minutes to about 30 minutes, about 30 minutes to about 1 hour, about 1 hour to about 2 hours, about 2 hours to about 4 hours, about 4 hours to about 6 hours, about 6 hours to about 8 hours, or about 8 hours to about 12 hours) before being removed.

Optionally, the starting nut material may be processed prior to being contacted with the supercritical fluid. For example, the starting nut material may be chopped, ground, pressed, or milled prior contacting the nut material with the supercritical fluid. The initial processing may extract a portion of the oil from the nut material, which can be further defatted using the supercritical fluid. In some embodiments, the nut material is pasteurized, for example by steam pasteurization or chemical pasteurization (for example, by using propylene oxide). In some embodiments, the nut material is unpasteurized.

In one embodiment, nuts are treated with supercritical fluid or critical liquid gas (e.g., CO₂) treatment for separation of fat from nut proteins. In some embodiments, the nuts are reduced in size (e.g., ground, finely chopped, etc.) prior to supercritical fluid treatment. In particular, nuts may include peanuts, or in other embodiments, one or more tree nuts (e.g., walnut, cashew, hazelnut, almond, pistachio, pecan, or other nuts). In other instances, previously defatted nut flour (e.g., 12% defatted peanut flour) can be further defatted using supercritical fluid CO₂ treatment (or other supercritical fluid) to yield an ultra-low fat, hyper-allergenic nut flour.

The nut flour or nut composition manufacturing protocols using supercritical fluid defatting treatment as described herein are expected to yield ultra-low fat, hyper-allergenic nut flours and compositions with stable nut proteins for use in oral immunotherapy protocols. In some examples, the supercritical fluid defatting treatment is expected to produce a peanut flour having less than about 12% fat (e.g., oil) content, less than about 10% fat content, less than about 8% fat content, less than about 6% fat content, less than about 5% fat content, between about 3% fat content and about 7% fat content, between about 3% fat content and about 5% fat content, or about 4% fat content following treatment. In other examples, the supercritical fluid defatting treatment is expected to produce a tree nut flour having less than about 12% fat (e.g., oil) content, less than about 10% fat content, less than about 8% fat content, less than about 6% fat content, less than about 5% fat content, between about 1% fat content and about 7% fat content, between about 1% fat content and about 5% fat content, about 2% fat content or about 3% fat content following treatment. In other examples, the supercritical fluid defatting process is expected to produce a nut flour between about 0.1 and about 1% fat content, about 0.5% fat content or 1% fat content. The starting nut material can be raw nuts, or in other embodiments, the starting nut material may be roasted.

In particular embodiments, supercritical fluid oil extraction from ground or finely ground nuts (e.g., passing through an 18 mesh sleeve) can include extraction at pressures from about 4000 psi to about 10.500 psi and at temperatures from about 17° C. to about 58° C. In certain embodiments, contact time between the supercritical fluid and the ground nut material can range from approximately 1 hour to about 5.5 hours per extraction cycle. One or more supercritical fluid extraction cycles can be performed until a desired fat (e.g., oil) content is achieved.

The defatted nut flour can be milled to produce a defatted nut flour with smaller sized particles. In some embodiments, the defatted nut flour is milled such that approximately all of the milled and defatted nut flour passes through a 2 mm (10 mesh) sieve or smaller, such as a 1 mm (18 mesh) sieve or smaller. The defatted nut flour can also be milled to produce a defatted nut flour with a desired particle size distribution. For example, in some embodiments, the defatted nut flour is milled such that about 25% or more, about 50% or more, about 75% or more, about 80% or more, about 85% or more, about 90% or more, about 95% or more, about 96% or more, about 97% or more, about 98% or more, about 99% or more, about 99.5% or more, or about 99.9% or more of the defatted nut flour by weight passes through a 2 mm (10 mesh) sieve, a 1 mm (18 mesh) sieve, a 841 µm (20 mesh) sieve, a 707 µm (25 mesh) sieve, a 595 µm (30 mesh) sieve, a 500 µm (35 mesh) sieve, a 420 µm (40 mesh) sieve, a 354 µm (45 mesh) sieve, a 354 µm (45 mesh) sieve, a 297 µm (50 mesh) sieve, or a 250 µm (60 mesh) sieve, a 210 µm (70 mesh) sieve, a 177 µm (80 mesh) sieve, or a 149 µm (100 mesh) sieve.

Larger particles can be separated from smaller particles to produce a defatted nut flour with an even smaller average particle sizes or a smaller particle size distribution. The larger particles can be separated from the smaller particles using, for example, one or more sieves. In some embodiments, the smaller particles are retained for further processing, such as formulating the nut flour into a pharmaceutical composition (for example by adding one or more carrier materials, such as a diluent, glidant, or lubricant), or packaging in a package (such as a capsule). In some embodiments, larger particles are separated from smaller particles such that approximately all of the defatted nut flour passes through a 1 mm (18 mesh) sieve, a 841 µm (20 mesh) sieve, a 707 µm (25 mesh) sieve, a 595 µm (30 mesh) sieve, a 500 µm (35 mesh) sieve, a 420 µm (40 mesh) sieve, a 354 µm (45 mesh) sieve, a 354 µm (45 mesh) sieve, a 297 µm (50 mesh) sieve, or a 250 µm (60 mesh) sieve, a 210 µm (70 mesh) sieve, a 177 µm (80 mesh) sieve, or a 149 µm (100 mesh) sieve.

Defatting of the nut flour using supercritical fluid can be repeated one or more times, which may further include one or more milling steps. For example, a nut material can be defatted by contacting the nut material with a supercritical fluid to produce a defatted nut flour, removing the supercritical fluid from the defatted nut flour, and re-contacting the defatted nut flour with fresh supercritical fluid. In some embodiments, the nut flour is milled to produce smaller sized particles prior to re-contacting the defatted nut flour with fresh supercritical fluid.

Standard testing of the extracted nut flour can be used to determine suitability for use in nut flour formulations and food products and for use in oral immunotherapy treatment regimes. The nut flour material may be tested for appearance, identity, total protein content and moisture content prior to release for formulation production (see, e.g., Table 5). The nut flour may be stored under controlled conditions at 2-8° C. In some embodiments, the nut flour is stored at about 2° C. to about 40° C., about 10° C. to about 35° C., about 15° C. to about 30° C., or about 20° C. to about 25° C. In some embodiments, the nut flour material is tested for particle size or particle size distribution, for example by passing the nut flour through one or more sieves.

In some embodiments, the oil content of the nut flour is measured, for example by measuring a mass or volume of the nut flour. The oil content of the nut flour can be measured, for example, using a hexane Soxhlet extraction method. In some embodiments, the total protein content of the defatted nut flour is measured, for example by using a Bradford assay.

Appearance assessments may be performed on the ground nut material and/or on the extracted protein nut flour (e.g., during one or more extraction steps and/or of the final ultra-low fat nut flour prior to formulation and/or encapsulation). Assessment of the appearance may include, for example, visually inspecting the container against a white background illuminated by a full spectrum light.

Moisture content may impact the stability of proteins, and understanding the changes in moisture content before and after extraction of oil and volatiles as well as over time is useful for understanding changes in a subsequently prepared formulation that may, in some instances, lead to shorter shelf life. Nut flour moisture content may be measured using Loss on Drying (LOD) determinations according to the USP. Conditions for the LOD may be determined based on requirements for the peanut flour and/or subsequently formulated excipients.

One or more nut protein allergens in the defatted nut material can be characterized. The one or more nut protein allergens in the defatted nut material may be characterized, for example, for the presence of one or more protein allergens, an amount of one or more protein allergens, an amount of one or more protein allergens relative to a different one or more protein allergens, or bioactivity of one or more protein allergens. The one or more protein allergens can be characterized, for example using an enzyme linked immunosorbent assay (ELISA), reversed-phase high performance liquid chromatography (RP-HPLC), size-exclusion chromatography (SEC), an immunoblot, mass spectrometry, liquid chromatography-mass spectrometry (LC-MS), LC-MS/MS, or any other known method. For example, an ELISA can be used to determine the bioactivity of one or more protein allergens, for example to determine whether the protein allergen was made non-allergenic during the defatting process. In some embodiments, RP-HPLC is used to determine the presence of or relative amounts of one or more protein allergens. Exemplary protein allergens that may be characterized include one or more peanut protein allergens (such as Ara h 1, Ara h 2, or Ara h 6) or walnut protein allergens (such as Jug r 1 or Jug r 2).

Reverse phase high performance liquid chromatography (RP-HPLC) may be used to physically separate peanut protein allergens (e.g., Ara h 1, Ara h 2, Ara-h 6) as described in U.S. Pat. No. 9,198,869 and U.S. Pat. Publication No. 2014/0271721, which are incorporated herein by reference in their entireties. RP-HPLC may be used to confirm identity of the ultra-low fat peanut flour as well as final formulation. Samples may be analyzed according to the methods described in more detail in U.S. Pat. Publication No. 2014/0271721.

Chromatographic analysis of samples extracted using the RP-HPLC method may produce a chromatographic “fingerprint” that is unique to peanut flour extracts. The protein content of each of these regions (mg/g) may be quantitated as follows:

The region of the samples that elute between approximately 12 minutes and 35 minutes may be integrated. The total area integrated may be quantitated against a BSA standard. The total extractable protein content may then calculated using the following equation:

$$\text{mg} / \text{g} \text{protein} = \frac{{R}_{u}}{{R}_{s}} \times {C}_{S T D} \times \frac{{V}_{S a m p l e}}{W {t}_{S a m p l e}}$$

where:
Rᵤ = Total Ara h Protein Peak Area or Ara h Species Peak Area in the Working Sample;
Rₛ = Average BSA Peak Area in all Working Standards CSTD = BSA Working Standard Concentration (mg/mL);
Vₛₐₘₚₗₑ = Total Diluent Volume of the Working Sample (10.0 mL); and
Wtₛₐₘₚᵢₑ = Weight of peanut flour sample (g).
Relative percent content of total protein for each region can then be calculated.

The defatted nut flour can be further processed for oral immunotherapy, for example by combining the defatted nut flour with one or more carrier materials (such as a glidant, a lubricant, or a diluent). The carrier materials combined with the defatted nut flour dilute the proteins in the nut flour, and the amount of carrier material (e.g., diluent) added to the nut flour based on a desired protein concentration. In some embodiments, a predetermined amount of protein is included in a dosage form. To obtain various dosage forms with different measured doses (i.e., different amounts of protein), the volume of the dosage form or the protein concentration in the formulated nut flour can be adjusted. In some embodiments, the defatted nut flour (which may include one or more carrier materials) is further processed by measuring a dose of the defatted nut flour, for example by measuring a volume or weight of defatted nut flour in a package. In some embodiments, the defatted nut flour is processed by packaging the defatted nut flour in a capsule, an envelope, a sachet, a pouch, a stick pack, or any other suitable packaging. In some embodiments, the packaging has a volume of about 5 mL or less, about 4 mL or less, about 3 mL or less, about 2 mL or less, or about 1 mL or less.

### IV. Compositions / Formulations

Provided herein are peanut and tree nut flour compositions and formulations, including encapsulated formulations and food-based chews for use in oral immunotherapy. In one embodiment, a nut flour composition comprises ultra-low fat nut flour extracting using supercritical fluid (e.g., _{S}CO₂). Formulations, including encapsulated formulations, comprise ultra-low fat nut flour blended with one or more excipients. For example, in addition to ultra-low fat nut flour, the formulations can comprise one or more of each of diluents, glidants, lubricants, colorants, and capsule shell components.

Smaller particle size of the defatted and milled nut flour allows for easier formulation and packaging, particularly when preparing low-dose dosage forms for oral immunotherapy. Because of the significant risk of a severe anaphylactic reaction due to administering a higher than desired dose of an oral immunotherapy formulation to a subject with an allergy, care must be taken to ensure the desired or intended dose is actually the dose administered. Therefore, variance in doses manufactured for oral immunotherapy should be minimized. Fat in non-defatted nut material causes particles to stick together, resulting in larger and difficult to formulate nut flour particles. By defatting nut material and milling the resulting defatted nut material, for example in accordance with the methods described herein, particle size of the nut flour can be substantially reduced.

Size and size distribution of the allergenic nut flour particles can be determined using one or more sieves. Sieves of a certain mesh size separate smaller particles (which pass through the sieve) from larger particles, and particles that pass through the sieve will be smaller than the sieve size. Sieves are often given in a “mesh” size which can be readily converted to standard units by a person of skill in the art. In some embodiments, approximately all of the defatted nut flour (i.e., tree nut flour or peanut flour) passes through a 1 mm (18 mesh) sieve, 420 µm (40 mesh) sieve, a 250 µm (60 mesh) sieve, a 177 µm (80 mesh) sieve, a 149 µm (100 mesh) sieve, or a 74 µm (200 mesh) sieve. In some embodiments, about 25% or more, about 50% or more, about 75% or more, about 80% or more, about 85% or more, about 90% or more, about 95% or more, about 96% or more, about 97% or more, about 98% or more, about 99% or more, about 99.5% or more, or about 99.9% or more of the defatted nut flour (i.e., defatted tree nut flour or defatted peanut flour) passes through a 1 mm (18 mesh) sieve, 420 µm (40 mesh) sieve, a 250 µm (60 mesh) sieve, a 177 µm (80 mesh) sieve, a 149 µm (100 mesh) sieve, or a 74 µm (200 mesh) sieve.

In some embodiments, defatted nut flour comprising one or more carrier materials (such as a glidant, lubricant, and/or diluent) results in decreased clumping of the nut flour particles compared to the defatted nut flour with the one or more carrier materials. In some embodiments, approximately all of the defatted nut flour comprising one or more carrier materials a 1 mm (18 mesh) sieve, 420 µm (40 mesh) sieve, a 250 µm (60 mesh) sieve, a 177 µm (80 mesh) sieve, a 149 µm (100 mesh) sieve, or a 74 µm (200 mesh) sieve. In some embodiments, bout 25% or more, about 50% or more, about 75% or more, about 80% or more, about 85% or more, about 90% or more, about 95% or more, about 96% or more, about 97% or more, about 98% or more, about 99% or more, about 99.5% or more, or about 99.9% or more of the defatted nut flour (i.e., defatted tree nut flour or defatted peanut flour) comprising one or more carrier materials (such as a glidant, lubricant, and/or diluent) passes through a 1 mm (18 mesh) sieve, 420 µm (40 mesh) sieve, a 250 µm (60 mesh) sieve, a 177 µm (80 mesh) sieve, a 149 µm (100 mesh) sieve, or a 74 µm (200 mesh) sieve.

The allergenic nut flour is preferably defatted compared to natural nut material. Defatting of the nut material facilitates milling of the nut material into a nut flour with small-sized particles. In some embodiments, the defatted nut flour (i.e., defatted tree nut flour or defatted peanut flour) has less than about 12% oil content by weight, less than about 10% oil content by weight, less than about 8% oil content by weight, less than about 6% oil content by weight, less than about 5% oil content by weight, less than about 4% oil content by weight, less than about 3% oil content by weight, less than about 2% oil content by weight, less than about 1% oil content by weight, or less than about 0.5% oil content by weight. In some embodiments, the defatted nut flour (i.e., defatted tree nut flour or defatted peanut flour) has an oil content between about 0.2% and about 0.5% by weight, between about 0.5% and about 1% by weight, between about 1% and about 2% by weight, between about 2% and about 3% by weight, between about 3% and about 4% by weight, between about 4% and about 5% by weight, between about 5% and about 6% by weight, between about 6% and about 8% by weight, between about 8% and about 10% by weight, or between about 10% and about 12% by weight.

In addition to a defatted nut flour, a pharmaceutical composition may include one or more carrier materials, such as one or more of a glidant, a diluent, or a lubricant. The carrier material can be blended with the nut flour to increase ease of handling of the pharmaceutical composition for manufacturing dosage forms.

Dosage forms for oral immunotherapy include a measured amount of a defatted nut flour. The nut flour is measured to a desired dose, and can be formatted for oral administration. In some embodiments, the nut flour, which may be combined with one or more carrier materials, is encapsulated in a capsule; sealed in an envelope, a sachet, a pouch, a stick pack, or other suitable packaging; compressed into a tablet (which may be a chewable tablet); or formatted in any other suitable packaging. In some embodiments, the packaging has a volume of about 5 mL or less, about 4 mL or less, about 3 mL or less, about 2 mL or less, or about 1 mL or less. The packaging may also be contained within a secondary packaging, such as a blister pack or box. The packaging (either the primary packaging or the secondary packaging) can identify the dosage (i.e., the amount of nut protein or the amount of nut flour) contained within the dosage form or package. In some embodiments, the capsule is made from a digestible material and can be swallowed whole. In some embodiments, the capsule, envelope, sachet, pouch, stick pack or other packaging is opened and the nut flour contained within the packaging is mixed with a food stuff for oral administration. For example, children adverse to or incapable of swallowing a capsule may find mixing the nut flour with pudding, oatmeal, or other food stuff allows for easier oral administration. In some embodiments, the food stuff is hypoallergenic food.

In particular embodiments, a composition comprising ultra-low fat nut flour (e.g., peanut flour, tree nut flour), may include food stuff for providing to a patient having nut allergies during the course of an oral immunotherapy regime. Non-limiting examples of food stuff comprising characterized nut flour contents, in accordance with aspects of the present technology, include baked items (e.g., cookies, crackers, food bars, etc.), food pellets, candy, mixing powders, etc. Other examples of food stuff include pudding and oats (such as oatmeal).

The formulation of chewable tablets is known in the art and takes into account qualities such as hardness, disintegration, dissolution, tablet size, thickness, friability and taste, which may impact the ability or willingness of a patient to chew the chewable tablet (i.e., a patient may swallow whole, rather than chew, a bad tasting tablet), bioavailability and bioequivalence. Various factors are involved in the formulation of chewable form factors. See Renu et al., Chewable Tablets: A Comprehensive Review. Pharma Innovation Journal 2015; 4(5): 100-105. The major formulation factors include flow, lubrication, disintegration, organoleptic properties, compressibility, compatibility, and stability. In oral immunotherapy, taste masking is very important since allergic individuals often have taste aversions to the foods they are allergic to. Product design and development considerations include: disintegrant(s) to facilitate release of the active ingredient, and sweeteners and flavoring agents for taste-masking. All components for an FDA approved product must contain excipients found in Handbook of Pharmaceutical Excipients, 6th Edition, August 2009. Editors: R.C. Rowe, P.J. Sheskey and M.E. Quinn. Publishers: The Pharmaceutical Press, London, UK; American Pharmaceutical Association, Washington DC, USA. ISBN: 978 0 85369 792 3 (UK) 978 1 58212 135 2 (USA). Examples of suitable formulation components are known in the art and may include those listed in Table 1.

TABLE 1

| | | |
|---|---|---|
| Category | Reason | Examples |
| API | Active ingredient | Nut flour |
| Bulking agent | Adds mass | Soluble fibers like guar gum and psyllium husk, Carnuba Wax, Glycerin, Beta Glucan, Mannitol, Maltitol, Polydextrose, Methylcellulose, and Pectin. |
| Thickeners and stabilizers | With emulsifiers, maintain the texture of food | Agars, Alginates, Carrageenans, Gum Arabic, Guar gum, Pectin, Starch, Sodium carboxymethyl cellulose |
| Sweeteners | Taste masking | Sugar, honey, maple syrup, corn syrup, Saccharin, Aspartame, Acesulfame potassium (Ace-K), Sucralose, Neotame, Advantame, Steviol glycosides, Luo Han Guo fruit extracts |
| Matrix | Hold ingredients together Makes for palatable mouth-feel | Fats: Coconut oil, sterotex, stearine, butter, oil |
| Flavors | Taste masking | FONO ingredients Citric acid |
| Colors | | Natural and artificial colors |
| Emulsifier | Mouth feel, Texture | Lecithin, Esters of monoglycerides of fatty acids, Mono- and diglycerides of fatty acids |
| Disintegrant¹ | Facilitates release of active ingredient | Starch, Corn Starch, or Potato Starch, Cross-linked polyvinylpyrrolidone, Modified Cellulose |

1. See www.carterpharmaceuticalconsulting.com/articles/The-role-of-disintergrants.html

In certain embodiments, including encapsulated formulations, the nut flour formulations may comprise one or more diluents, one or more glidants, and/or one or more lubricants. Intended dosage forms of nut flour formulations comprising the ultra-low fat nut flours described herein, may include, for example, a (hydroxypropyl)methyl cellulose (HPMC) based capsule, and the strength of the dosage forms may be about 0.2 mg, about 0.5 mg, about 1 mg, about 10 mg, about 100 mg, about 475 mg, about 1000 mg of nut protein or other amount of nut protein. Nut protein (e.g., nut flour) may, in some instances, be a cohesive material without inherent flow properties conducive to conventional pharmaceutical manufacturing processes. Thus, inactive pharmaceutical ingredients (excipients) may be added to the formulation so the nut flour may be developed into a pharmaceutical dosage form with flow characteristics to enhance both manufacturing and also delivery of the dosage form.

Under cGMP manufacturing conditions, the nut flour is formulated with a diluent, a glidant, and/or a lubricant, and is subsequently encapsulated as 0.2, 0.5, 1, 10, 100, 475, 500 or 1000 mg of nut flour in size 3, 00 or 000 (hydroxypropyl)methyl cellulose (HPMC) capsules.

In particular formulations, a diluent provides the opportunity to formulate the low and high doses to contain adequate volume for dispersal from the opened capsule. The glidant and lubricant can add flowability to the nut flour such that the capsule is easily emptied of flour by the subject. For clinical trials, the capsules will be bulk packed into amber colored bottles or into blister packs. At the time of use, capsule(s) will be opened and the content mixed into taste-masking food immediately prior to administration.

In one embodiment, a composition comprises one or more diluents. “Diluents” for use in the formulations include, but are not limited to, alginic acid and salts thereof; cellulose derivatives such as carboxymethylcellulose, methylcellulose (e.g., a cellulose ether sold under the trademane METHOCEL™), hydroxypropylmethylcellulose, hydroxyethylcellulose, hydroxypropylcellulose (e.g., a hydroxyporopylcellulose sold under the tradename KLUCEL™) ethylcellulose (e.g., an ethylcellulose sold under the tradename ETHOCEL™), microcrystalline cellulose (e.g., a microcrystalline cellulose sold under the trademname AVICEL®); silicified microcrystalline cellulose; microcrystal- line dextrose; amylase; magnesium aluminum silicate; polysaccharide acids; bentonites; gelatin; polyvinylpyrrolidone/vinyl acetate copolymer; crosspovidone; povidone; starch; pregelatinized starch; tragacanth, dextrin, a sugar, such as sucrose (e.g., a sucrose sold under the tradename DI-PAC®), glucose, dextrose, molasses, mannitol, sorbitol, xylitol (e.g., a xylitol sold under the tradename XYLITAB®), lactose (e.g., lactose monohydrate, lactose anhydrous, etc.); dicalcium phosphate; a natural or synthetic gum such as acacia, tragacanth, ghatti gum, mucilage of isapol husks, polyvinylpyrrolidone (e.g., a polyvinylpyrrolidone sold under any one of the tradenames POLYVIDIONE® CL, KOLLIDON® CL, POLYPLASDONE® XL-10), larch arabogalactan, a magnesium aluminum silicate such as a material sold under the tradename VEEGUM®, polyethylene glycol, waxes, sodium alginate, a starch, e.g., a natural starch such as corn starch or potato starch, a pregelatinized starch such as a starch sold under any one of the tradenames STARCH 1500® (Colorcon), NATIONAL™ 1551 or AMIJEL®, or sodium starch glycolate such as a material sold under the tradename PROMOGEL® or EXPLOTAB®; a cross-linked starch such as sodium starch glycolate; a cross-linked polymer such as crospovidone; a cross-linked polyvinylpyrrolidone; alginate such as alginic acid or a salt of alginic acid such as sodium alginate; a clay such as a material sold under the tradename VEEGUM® HV (magnesium aluminum silicate); a gum such as agar, guar, locust bean, Karaya, pectin, or tragacanth; sodium starch glycolate; bentonite; a natural sponge; a surfactant; a resin such as a cation-exchange resin; citrus pulp; sodium lauryl sulfate; sodium lauryl sulfate in combination starch; and combinations thereof. In one embodiment, the formulation comprises microcrystalline cellulose or STARCH 1500®. In another embodiment, the formulation comprises microcrystalline cellulose and STARCH 1500®.

Suitable glidants (anti-caking agents) for use in the solid dosage forms described herein include, but are not limited to, colloidal silicon dioxide (for example, a colloidal silicon dioxide sold under the trademark CAB-O-SIL®) and talc (e.g., Ultra Talc 4000). In one embodiment, the composition comprises the colloidal silicon dioxide sold under the trademark CAB-O-SIL®.

Suitable lubricants for use in the solid dosage forms described herein include, but are not limited to, stearic acid, calcium hydroxide, talc, corn starch, sodium stearyl fumerate, alkali-metal and alkaline earth metal salts, such as aluminum, calcium, magnesium, zinc, stearic acid, sodium stearates, magnesium stearate, zinc stearate, waxes, a blend of magnesium stearate and sodium lauryl sulfate such as the material sold under the tradename STEAR-0-WETTm, boric acid, sodium benzoate, sodium acetate, sodium chloride, leucine, a polyethylene glycol or a methoxypolyethylene glycol such as a polyethylene glycol sold under the tradename CARBOWAX™, PEG 4000, PEG 5000, PEG 6000, propylene glycol, sodiumoleate, glyceryl behenate, glyceryl palmitostearate, glyceryl benzoate, magnesium or sodium lauryl sulfate, and combinations thereof. In one embodiment, the composition comprises magnesium stearate. In another embodiment, the composition comprises sodium stearyl fumerate.

In some embodiments, a formulation may further comprise one or more filling agents. “Filling agents” include compounds such as lactose, calcium carbonate, calcium phosphate, dibasic calcium phosphate, calcium sulfate, microcrystalline cellulose, cellulose powder, dextrose, dextrates, dextran, starches, pregelatinized starch, sucrose, xylitol, lactitol, mamiitol, sorbitol, sodium chloride, polyethylene glycol, and combinations thereof.

In one embodiment, a composition described herein comprises nut proteins in a concentration from about 0.05% to about 100% w/w, or any integer therein. In another embodiment, a composition described herein comprises one or more nut proteins in a concentration from about 0.1% to about 100% w/w. In another embodiment, a composition described herein comprises one or more nut proteins in a concentration from about 0.5%, about 1%, about 2%, about 4%, or about 100% w/w. In another embodiment, a composition described herein comprises one or more nut proteins in a concentration from about 0.7%, about 1.42%, about 3.93%, or about 100% w/w.

In one embodiment, a composition described herein comprises one or more nut proteins in a target unit weight from about 0.5 mg/capsule to about 1100 mg/capsule, or any integer therein. In yet another embodiment, a composition described herein comprises one or more nut proteins in a target unit weight from about 1.0 mg/capsule to about 1000 mg/capsule. In yet another embodiment, a composition described herein comprises one or more nut proteins in a target unit weight of about 1.0 mg/capsule to about 2 mg/capsule, about 3 mg/capsule, about 6 mg/capsule, about 12 mg/capsule, about 20 mg/capsule, about 40 mg/capsule, about 80 mg/capsule, about 120 mg/capsule, about 160 mg/capsule, about 200 mg/capsule, about 240 mg/capsule, about 300 mg/capsule, about 500 mg/capsule or about 1000 mg/capsule.

In some embodiments, the dosage form includes a measured amount of a defatted nut flour such that the dosage form includes about 0.1 mg to about 2000 mg nut protein (such as about 0.1 mg to about 0.5 mg nut protein, about 0.5 mg to about 1 mg nut protein, about 1 mg to about 2.5 mg nut protein, about 2.5 mg to about 5 mg nut protein, about 5 mg to about 10 mg nut protein, about 10 mg to about 25 mg nut protein, about 25 mg to about 50 mg nut protein, about 50 mg to about 100 mg nut protein, about 100 mg to about 250 mg nut protein, about 250 mg to about 500 mg nut protein, or about 500 mg to about 1000 mg nut protein).

The concentration of diluent in a composition described herein may be from about 1% to about 99% w/w of the composition. In one embodiment, the concentration of diluent in a composition described herein may be from about 10% to about 90% w/w of the composition. For example, the diluent may be STARCH 1500® and the concentration may be about 9.86% to about 10% w/w of the composition. The target unit weight of the diluent may be from about 10 to about 60 mg/capsule. For example, the diluent may be STARCH 1500® and the target unit weight may be about 14, about 14.5, or about 52.5 mg/capsule.

In one embodiment, the diluent may be microcrystalline cellulose and the concentration may be about 90% to about 60% w/w of the composition. For example, the diluent may be microcrystalline cellulose and the concentration may be about 88.66%, about 87.58%, about 85.07%, or about 65.66% w/w of the composition. In one embodiment, the target unit weight of the diluent may be from about 100 to about 410 mg/capsule. For example, the diluent may be microcrystalline cellulose and the target unit weight may be about 125.55, about 126.99, about 446.61, or about 394 mg/capsule.

The concentration of glidant in a composition described herein may be from about 0.01% to about 10% w/w of the composition. In one embodiment, the glidant is Cab-0- Sil and the concentration of glidant in a composition described herein may be about 0.01%, about 0.05%, about 0.1%, about 0.25%, about 0.5%, about 0.75%, about 1.0%, about 1.25%, or about 1.5% w/w of the composition. The target unit weight of the glidant may be from about 0.05 to about 5 mg/capsule. In one embodiment, the glidant is Cab-0-Sil and the target unit weight is about 0.725, about 2.625 or about 3.0 mg/capsule.

The concentration of lubricant in a composition described herein may be from about 0.01% to about 10% w/w of the composition. In one embodiment, the lubricant is magnesium stearate and the concentration of lubricant in a composition described herein may be about 0.01%, about 0.05%, about 0.1%, about 0.25%, about 0.5%, about 0.75%, about 1.0%, about 1.25%, or about 1.5% w/w of the composition. The target unit weight of the lubricant may be from about 0.05 to about 5 mg/capsule. In one embodiment, the lubricant is magnesium stearate and the target unit weight is about 0.725, about 2.625 or about 3.0 mg/capsule.

It will be understood that quantitative formulas will be adjusted depending on manufacturing fill weights. Final fill weights may vary from about 150 mg to about 600 mg to about 1000 mg. In one embodiment, a formulation comprising ultra-low fat nut flour containing about 0.5 mg nut protein is manufactured with a final fill weight of about 158 mg. In another embodiment, a nut protein formulation containing about 1.0 mg nut protein is manufactured with a final fill weight of about 150 mg. In another embodiment, a nut flour formulation containing about 10.0 mg nut protein is manufactured with a final fill weight of about 450 mg. In another embodiment, a nut protein formulation containing about 100 mg nut protein is manufactured with a final fill weight of about 600 mg.

In some embodiments, solid dosage forms may be in the form of a tablet, (including a suspension tablet, a fast-melt tablet, a bite-disintegration tablet, a rapid-disintegration tablet, an effervescent tablet, or a caplet), a pill, a powder (including a sterile packaged powder (such as a “sachet pack” or foil pouch), a dispensable powder, or an effervescent powder) a capsule (including both soft or hard capsules, e.g., capsules made from animal-derived gelatin or plant-derived HPMC, or “sprinkle capsules”), solid dispersion, solid solution, pellets, or granules. In other embodiments, the formulation is in the form of a powder. Additionally, formulations may be administered as a single capsule or in multiple capsule dosage form. In some embodiments, the formulation is administered in two, or three, or four, capsules or tablets or powder packages.

In some embodiments, solid dosage forms, e.g., tablets, effervescent tablets, and capsules, are prepared by mixing nut flour comprising characterized nut allergens with one or more pharmaceutical excipients to form a bulk blend composition. When referring to these bulk blend compositions as homogeneous, it is meant that the particles are dispersed evenly throughout the composition so that the composition may be readily subdivided into equally effective unit dosage forms, such as tablets, pills, and capsules. The individual unit dosages may also comprise film coatings, which disintegrate upon oral ingestion or upon contact with diluent. These formulations can be manufactured by conventional pharmacological techniques.

Conventional pharmacological techniques include, e.g., one or a combination of methods: (1) dry mixing, (2) direct compression, (3) milling, (4) dry or non-aqueous granulation, (5) wet granulation, or (6) fusion. See, e.g., Lachman et al., The Theory and Practice of Industrial Pharmacy (1986). Other methods include, e.g., spray drying, pan coating, melt granulation, granulation, fluidized bed spray drying or coating (e.g., Wurster coating), tangential coating, top spraying, tableting, extruding and the like.

The pharmaceutical solid dosage forms described herein can comprise the compositions described herein and one or more pharmaceutically acceptable additives such as a compatible carrier, binder, filling agent, suspending agent, flavoring agent, sweetening agent, disintegrating agent, dispersing agent, surfactant, lubricant, colorant, diluent, solubilizer, moistening agent, plasticizer, stabilizer, penetration enhancer, wetting agent, anti-foaming agent, antioxidant, preservative, or one or more combination thereof. In still other aspects, using standard coating procedures, such as those described in Remington’s Pharmaceutical Sciences, 20th Edition (2000), a film coating is provided around the formulation. In one embodiment, some or all of the particles are coated. In another embodiment, some or all of the particles are microencapsulated. In yet another embodiment, some or all of the nut allergens are amorphous material coated and/or microencapsulated with inert excipients. In still another embodiment, the particles are not microencapsulated and are uncoated.

Compressed tablets are solid dosage forms prepared by compacting the bulk blend formulations described above. In various embodiments, compressed tablets which are designed to dissolve in the mouth will comprise one or more flavoring agents. In other embodiments, the compressed tablets will comprise a film surrounding the final compressed tablet. In some embodiments, the film coating can provide a delayed release of the formulation. In other embodiments, the film coating aids in subject compliance (e.g., a coating aid sold under the tradename OPADRY® coatings or sugar coating). Film coatings comprising OPADRY® typically range from about 1% to about 3% of the tablet weight. In other embodiments, the compressed tablets comprise one or more excipients.

A capsule may be prepared, e.g., by placing the bulk blend formulation, described above, inside of a capsule. In some embodiments, the formulations (non-aqueous suspensions and solutions) are placed in a soft gelatin capsule. In other embodiments, the formulations are placed in standard gelatin capsules or non-gelatin capsules such as capsules comprising HPMC. In other embodiments, the formulations are placed in a sprinkle capsule, wherein the capsule may be swallowed whole or the capsule may be opened and the contents sprinkled on food prior to eating. In some embodiments of the present invention, the therapeutic dose is split into multiple (e.g., two, three, or four) capsules. In some embodiments, the entire dose of the formulation is delivered in a capsule form.

In various embodiments, the particles and one or more excipients are dry blended and compressed into a mass, such as a tablet, having a hardness sufficient to provide a pharmaceutical composition that substantially disintegrates within less than about 30 minutes, less than about 35 minutes, less than about 40 minutes, less than about 45 minutes, less than about 50 minutes, less than about 55 minutes, or less than about 60 minutes, after oral administration, thereby releasing the formulation into the gastrointestinal fluid.

In one aspect of the present invention, dosage forms may include microencapsulated formulations. In some embodiments, one or more other compatible materials are present in the microencapsulation material. Exemplary materials include, but are not limited to, pH modifiers, erosion facilitators, anti-foaming agents, antioxidants, flavoring agents, and carrier materials such as binders, suspending agents, disintegration agents, filling agents, surfactants, solubilizers, stabilizers, lubricants, wetting agents, and diluents.

Materials useful for the microencapsulation described herein include materials compatible with nut allergens which sufficiently isolate nut allergens from other non-compatible excipients. Materials compatible with nut allergens are those that delay the release of the nut allergens in vivo.

Exemplary microencapsulation materials useful for delaying the release of the formulations include, but are not limited to, hydroxypropyl cellulose ethers (HPC) such as a hydorxypropyl cellulose ether sold under the tradename KLUCEL™ or Nissa HPC, low-substituted hydroxypropyl cellulose ethers (L-HPC), hydroxypropyl methyl cellulose ethers (HPMC) such as an material sold under the tradename SEPIFILM™-LC, PHARMACOAT®,METOLOSE® SR, METHOCEL™-E, OPADRY® VS, Prima Flo, BENECEL™ MP824, and BENECEL™MP843, methylcellulose polymers such as a material sold under the tradename METHOCEL™-A, hydroxypropylmethylcellulose acetate stearate Aqoat (HF-LS, HF-LG, HF-MS) and METOLOSE®, Ethylcelluloses (EC) and mixtures thereof such as E461, ETHOCEL™, AQUALON™-EC, SURELEASE®, Polyvinyl alcohol (PVA) such as OPADRY® AMB, hydroxyethylcelluloses such as NATROSOL™, carboxymethylcelluloses and salts of carboxymethylcelluloses (CMC) such as AQUALON™-CMC, polyvinyl alcohol and polyethylene glycol co-polymers such as KOLLICOAT® IR, monoglycerides (Myverol), triglycerides (KLX), polyethylene glycols, modified food starch, acrylic polymers and mixtures of acrylic polymers with cellulose ethers such as any one of those sold under the tradename EUDRAGIT® EPO, EUDRAGIT® L300-55, EUDRAGIT® FS 300 EUDRAGIT® L100-55, EUDRAGIT® L100, EUDRAGIT® 5100, EUDRAGIT® R0100, EUDRAGIT® E100, EUDRAGIT® L12.5, EUDRAGIT® 512.5, EUDRAGIT® NE300, and EUDRAGIT® NE 400, cellulose acetate phthalate, sepifilms such as mixtures of HPMC and stearic acid, cyclodextrins, and mixtures of these materials.

Microencapsulated nut allergens may be formulated by methods known by one of ordinary skill in the art. Such known methods include, e.g., spray drying processes, spinning disk-solvent processes, hot melt processes, spray chilling methods, fluidized bed, electrostatic deposition, centrifugal extrusion, rotational suspension separation, polymerization at liquid-gas or solid-gas interface, pressure extrusion, or spraying solvent extraction bath. In addition to these, several chemical techniques, e.g., complex coacervation, solvent evaporation, polymer-polymer incompatibility, interfacial polymerization in liquid media, in situ polymerization, in-liquid drying, and desolvation in liquid media could also be used. Furthermore, other methods such as roller compaction, extrusion/spheronization, coacervation, or nanoparticle coating may also be used.

The formulations described herein are administered and dosed in accordance with good medical practice, taking into account the clinical condition of the individual subject, the site and method of administration, scheduling of administration, and other factors known to medical practitioners.

The dosage forms described herein may be included in a kit, which further includes instructions for use in oral immunotherapy. The instructions can include, for example, instructions for administration of the dosage form, such as instructions for daily administration of the dosage form or instructions for combining the defatted nut flour with a food stuff. In some embodiments, the instructions include instructions for treatment of an allergy or for oral immunotherapy, as described herein.

### V. Methods of Use

The formulations described herein may be used in oral immunotherapy (OIT) to treat a subject suffering from a nut allergy. For example, a subject may suffer from one or more of a peanut allergy or a tree nut (e.g., walnut, cashew, hazelnut, almond, pistachio, pecan, or other nuts) allergy.

In some embodiments, the subject is a human. In some embodiment, the subject is a human about 18 years of age or younger, such as about 16 years of age or younger, about 14 years of age or younger, about 12 years of age or younger, about 10 years of age or younger, about 9 years of age or younger, about 8 years of age or younger, about 7 years of age or younger, about 6 years of age or younger, about 5 years of age or younger, about 4 years of age or younger, about 3 years of age or younger, about 2 years of age or younger, or about 1 year of age or younger.

In some embodiments, a method of treating a tree nut allergy in a subject includes orally administering to the subject an effective amount of a pharmaceutical composition that includes defatted nut flour (such as defatted peanut flour or defatted tree nut flour). The defatted nut flour can be produced, for example, using the methods described herein, which can include contacting a nut material with a supercritical fluid to defat nut material. Production of the defatted nut material may also include milling the defatted nut flour and/or formulating the defatted nut flour with a carrier material. In some embodiments, the effective amount of the pharmaceutical composition includes about 0.1 mg to about 2000 mg of nut protein (such as tree nut protein), or any other measured amount as described herein.

A subject treated with the formulations described herein may exhibit a decreased anaphylactic reaction, relating to a decrease in clinical symptoms following treatment of symptoms associated with exposure to an anaphylactic allergen, which can involve exposure via cutaneous, respiratory, gastrointestinal, and mucosal (e.g., ocular, nasal, and aural) surfaces or a subcutaneous injection (e.g., via a bee sting) following treatment. In one embodiment, a subject may exhibit a decreased anaphylactic reaction of about 2%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90% or more compared to a subject receiving a placebo or a subject not receiving treatment.

A subject treated with a composition described herein may exhibit a decreased humoral response and/or T cell response following treatment. In one embodiment, a subject may exhibit a decreased humoral response and/or T cell response of about 2%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90% or more compared to a subject receiving a placebo or a subject not receiving treatment.

A subject treated with a composition described herein may exhibit a decreased IgE response and/or a decreased mast cell response following treatment. In one embodiment, a subject may exhibit a decreased IgE response and/or a decreased mast cell response of about 2%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90% or more compared to a subject receiving a placebo or a subject not receiving treatment.

A subject treated with the formulation may also exhibit an increased IgG4 response which replaces the IgE antibodies and tempers the immune response to allergens thus lessening the likelihood of an allergic reaction.

A subject treated with the formulations described herein may be better able to withstand an oral food challenge (OFC) following treatment.

A subject treated with a composition described herein may be desensitized to nut allergen following treatment. In one embodiment, a subject may be desensitized by about 2%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90% or more compared to a subject receiving a placebo or a subject not receiving treatment.

The compositions described herein may be administered in an escalation schedule. In one embodiment, escalating doses are administered to the subject on day 1 of treatment. For example, a subject may be administered, 1, 2, 3, 4, or 5 doses of a composition described herein on day 1. In another example, a subject may be administered 5 doses of a composition described herein in 30 minute increments on day 1. Subjects return on day 2 and receive a maximum tolerated dose. Subjects with moderate symptoms observed on day 2 may return on day 3 for the next lower dose under observation in a monitored clinic setting. Subjects able to withstand treatment on the initial day of treatment may be administered one or more further doses of a composition described herein.

In some embodiments, the effective amount of the composition is administered daily. In some embodiments, the same effective amount composition (i.e., same dose of the composition) is administered daily for a desired period of time, such as one or more weeks, two or more weeks, three or more weeks, or four or more weeks. In some embodiments, the dose of the composition is increase after the desired period of time. For example, the dose of the composition can be increased approximately once per week, approximately once per two weeks, approximately once per three weeks, or approximately once per month. The composition can be administered daily for the course of therapy at the same or at a different dose, which may be at least a month, at least two months, at least three months, at least four months, at least five months, or at least six months.

In one embodiment, a subject is further administered 1, 2, 3, 4, 5, 6, 7, 8, or 9 additional escalating doses of a composition described herein. The additional escalating doses may be administered to a subject in two-week intervals.

Following the final administration, the subject may, in some instances, be subject to an oral food challenge to determine if the subject has been desensitized to nut allergy.

In one embodiment of such methods, immediately prior to administration, an encapsulated capsule formulation may be broken apart and the ingredients mixed into taste -masking food.

In another embodiment, subjects continue taking active treatment for a 3-, 6-, 12-, 24-month or longer maintenance period.

Subjects may be monitored for onset of systemic symptoms including, for example, flushing, intensive itching on the skin, and sneezing and runny nose. Sense of heat, general discomfort and agitation/anxiety may also occur.

In one embodiment, the formulations provided herein are administered one or more days to a subject suffering from a nut allergy.

In one embodiment, the subject is able to increase the amount of protein they can consume without an allergic reaction by at least about 100% compared to a subject administered a placebo or not receiving treatment.

In another embodiment, the subject exhibits a reduced humoral response and/or a reduced T cell response.

In another embodiment, the subject exhibits reduced anaphylaxis, a reduced mast cell response, a reduced IgE response, reduced hives, or a combination thereof.

In some embodiments, a formulation provided herein may be administered in conjunction with a food product.

A subject may be administered 1, 2, 3, 4, or 5 doses of a formulation provided herein on the first day of treatment.

In one embodiment, a subject is administered 10 doses on the first day of treatment.

In another embodiment, the subject is administered said doses in 30 minute intervals.

The method may, in some instances further comprise one or more additional treatments.

In some embodiments, the one or more additional treatments comprise administration of a composition in two-week intervals.

In other embodiments, the one or more additional treatments comprise 1, 2, 3, 4, 5, 6, 7, 8, 9, or more doses of a composition.

In one embodiment, the method can further comprise administering an oral food challenge (OFC) following completion of the treatment regimen.

### VI. Combination Therapies

The formulations and methods described herein may also be used in conjunction with other well-known therapeutic compounds that are selected for their particular usefulness against the condition that is being treated. In general, the formulations described herein and, in embodiments where combinational therapy is employed, other compounds, do not have to be administered in the same formulation, and may, because of different physical and chemical characteristics, have to be administered by different routes, or they may be combined in a single formulation. The determination of the mode of administration and the advisability of administration, where possible, in the same formulation, is well within the knowledge of the skilled clinician. The initial administration can be made according to established protocols known in the art, and then, based upon the observed effects, the dosage, modes of administration and times of administration can be modified by the skilled clinician.

The particular choice of compounds used will depend upon the diagnosis of the attending physicians and their judgment of the condition of the subject and the appropriate treatment protocol. The compounds may be administered concurrently (e.g., simultaneously, essentially simultaneously or within the same treatment protocol) or sequentially, depending upon the condition of the subject, and the actual choice of compounds used. The determination of the order of administration, and the number of repetitions of administration of each therapeutic agent during a treatment protocol, is well within the knowledge of the skilled physician after evaluation of the severity of nut allergy being treated and the condition of the subj ect.

It is understood that the dosage regimen to treat, prevent, or ameliorate nut allergy, can be modified in accordance with a variety of factors. These factors include the age, weight, sex, diet, and/or medical condition of the subject. Thus, the dosage regimen actually employed can vary widely and therefore can deviate from the dosage regimens set forth herein.

The time period between the multiple administration steps may range from, a few minutes to several hours, depending upon the properties of each pharmaceutical agent, such as potency, solubility, bioavailability, plasma half-life and kinetic profile of the pharmaceutical agent. Circadian variation of the target molecule concentration may also determine the optimal dose interval.

In some embodiments, the formulation is administered with at least one other antihistamine agent, corticosteroid, beta agonist, anti-inflammatory agent, an anti-IgE antibody (e.g., omalizumab) and/or epinephrine.

### VII. Exemplary Embodiments

Embodiment 1. A tree nut flour composition comprising defatted tree nut flour, wherein at least 50% of the defatted tree nut flour by weight passes through a 250 µm sieve.

Embodiment 2. The tree nut flour composition of embodiment 1, wherein the defatted tree nut flour has an oil content of less than about 12% by weight.

Embodiment 3. The tree nut flour composition of embodiment 1, wherein the defatted tree nut flour has an oil content of less than about 6% by weight.

Embodiment 4. The tree nut flour composition of any one of embodiments 1-3, wherein the tree nut is walnut, almond, pecan, cashew, hazelnut, pine nut, brazil nut, or pistachio.

Embodiment 5. The tree nut flour composition of any one of embodiments 1-4, wherein approximately all of the defatted tree nut flour passes through a 1 mm sieve.

Embodiment 6. The tree nut flour composition of any one of embodiments 1-5, wherein approximately all of the defatted tree nut flour passes through a 250 µm sieve.

Embodiment 7. The tree nut flour composition of any one of embodiments 1-6, wherein approximately all of the defatted tree nut flour passes through a 149 µm sieve.

Embodiment 8. The tree nut flour composition of any one of embodiments 1-7, wherein approximately all of the defatted tree nut flour passes through a 74 µm sieve.

Embodiment 9. The tree nut flour composition of any one of embodiments 1-8, further comprising a carrier material.

Embodiment 10. The tree nut flour composition of embodiment 6, wherein the carrier material comprises one or more diluents, glidants, or lubricants.

Embodiment 11. The tree nut flour composition of any one of embodiments 1-10, wherein the defatted tree nut flour is produced according to a method comprising:
contacting a tree nut material with a supercritical fluid; and
milling the tree nut material to form the defatted tree nut flour.

Embodiment 12. The tree nut flour composition of embodiment 8, wherein the supercritical fluid is supercritical carbon dioxide.

Embodiment 13. The tree nut flour composition of any one of embodiments 1-12, wherein the tree nut flour composition is combined with a food stuff.

Embodiment 14. A method of treating nut allergy in a subject, comprising orally administering to the subject an effective amount of the tree nut flour composition according to any one of embodiments 1-13.

Embodiment 15. A dosage form for oral immunotherapy, comprising the tree nut flour composition according to any one of embodiments 1-13.

Embodiment 16. The dosage form of embodiment 15, comprising a measured amount of the tree nut flour composition.

Embodiment 17. The dosage form of embodiment 15 or 16, wherein the dosage form comprises about 0.1 mg to about 2000 mg of tree nut flour.

Embodiment 18. The dosage form of any one of embodiments 15-17, wherein the measured dose comprises about 0.5 mg to about 1000 mg nut protein.

Embodiment 19. The dosage form of any one of embodiments 15-18, wherein the tree nut flour composition is enclosed in a package.

Embodiment 20. The dosage form of embodiment 19, wherein the package identifies an amount of nut protein or an amount of nut flour contained within the dosage form or package.

Embodiment 21. The dosage form of any one of embodiments 15-20, wherein the tree nut flour composition is encapsulated in a capsule.

Embodiment 22. A dosage form for oral immunotherapy, comprising a measured amount of a defatted nut flour, wherein the defatted nut flour is produced according to a method comprising:
contacting a nut material with a supercritical fluid, thereby reducing the oil content of the nut material to form a defatted nut flour; and
measuring a dose of the defatted nut flour.

Embodiment 23. The dosage form of embodiment 22, wherein the measured dose comprises about 0.1 mg to about 2000 mg nut protein.

Embodiment 24. The dosage form of embodiment 22 or 23, wherein the measured dose comprises about 0.5 mg to about 1000 mg nut protein.

Embodiment 25. The dosage form of any one of embodiments 22-25, wherein the method of producing the defatted nut flour further comprises milling the defatted nut flour.

Embodiment 26. The dosage form of any one of embodiments 22-25, wherein the method of producing the defatted nut flour further comprises pressing or milling the nut material prior to contacting the nut material with the supercritical fluid.

Embodiment 27. The dosage form of any one of embodiments 22-26, wherein the defatted nut flour is combined with a carrier material.

Embodiment 28. The dosage form of embodiment 27, wherein the carrier material comprises one or more diluents, glidants, or lubricants.

Embodiment 29. The dosage form of any one of embodiments 22-28, wherein the defatted nut flour is enclosed in a package.

Embodiment 30. The dosage form of embodiment 29, wherein the package identifies an amount of nut protein or an amount of nut flour contained within the dosage form or package.

Embodiment 31. The dosage form of any one of embodiments 22-30, wherein the defatted nut flour is encapsulated in a capsule.

Embodiment 32. The dosage form of any one of embodiments 22-31, wherein at least 50% of the defatted nut flour by weight passes through a 250 µm sieve.

Embodiment 33. The dosage form of any one of embodiments 22-32, wherein approximately all of the defatted nut flour passes through a 1 mm sieve.

Embodiment 34. The dosage form of any one of embodiments 22-33, wherein approximately all of the defatted tree nut flour passes through a 250 µm sieve.

Embodiment 35. The dosage form of any one of embodiments 22-34, wherein approximately all of the defatted tree nut flour passes through a 149 µm sieve.

Embodiment 36. The dosage form of any one of embodiments 22-35, wherein approximately all of the defatted tree nut flour passes through a 74 µm sieve.

Embodiment 37. The dosage form of any one of embodiments 22-36, wherein the defatted nut flour has an oil content of less than about 12%.

Embodiment 38. The dosage form of any one of embodiments 22-37, wherein the defatted nut flour has an oil content of less than about 6%.

Embodiment 39. The dosage form of any one of embodiments 22-38, wherein the defatted nut flour is a peanut flour.

Embodiment 40. The dosage form of any one of embodiments 22-38, wherein the defatted nut flour is a tree nut flour.

Embodiment 41. The dosage form of embodiments 40, wherein the tree nut is walnut, almond, pecan, cashew, hazelnut, pine nut, brazil nut, or pistachio.

Embodiment 42. The dosage form of any one of embodiments 22-41, wherein the supercritical fluid is supercritical carbon dioxide.

Embodiment 43. A kit comprising the dosage form of any one of embodiment 15-42 and instructions for use in oral immunotherapy.

Embodiment 44. The kit of embodiment 43, wherein the instructions for use comprise instructions for combining the defatted nut flour with a food stuff.

Embodiment 45. The kit of embodiment 43 or 44, wherein the instructions for use comprise instructions for daily administration of the dosage form.

Embodiment 46. A method of manufacturing an ultra-low fat nut material comprising:
contacting a nut material having an initial oil content with a supercritical fluid to provide a defatted nut material having a reduced oil content; and
milling the defatted nut material.

Embodiment 47. A method of manufacturing an ultra-low fat nut material comprising:
contacting a nut material having an initial oil content with a supercritical fluid to provide a defatted nut material having a reduced oil content; and
measuring a dose of the defatted nut material for oral immunotherapy.

Embodiment 48. The method of embodiment 47, wherein the measured dose of the defatted nut material comprises about 0.1 mg to about 1000 mg nut protein.

Embodiment 49. A method of manufacturing an ultra-low fat nut material comprising:
contacting a nut material having an initial oil content with a supercritical fluid to provide a defatted nut material having a reduced oil content; and
packaging the defatted nut material in a package.

Embodiment 50. The method of embodiment 49, wherein the package has a volume of less than 5 mL.

Embodiment 51. The method of embodiment 49 or 50, wherein the package is a capsule.

Embodiment 52. The method of any one of embodiments 46-51, comprising combining the defatted nut material with a carrier material.

Embodiment 53. The method of embodiment 52, wherein the carrier material comprises one or more diluents, glidants, or lubricants.

Embodiment 54. The method of any one of embodiments 46-53, wherein the defatted nut material has an oil content between 0.1% and 12%.

Embodiment 55. The method of any one of embodiments 46-54, wherein the supercritical fluid is carbon dioxide.

Embodiment 56. The method of any one of embodiments 46-55, wherein the nut material is a peanut material.

Embodiment 57. The method of any one of embodiments 46-55, wherein the nut material is a tree nut material.

Embodiment 58. The method of embodiment 57, wherein the tree nut material is a walnut material, a cashew material, a hazelnut material, an almond material, a pistachio material, a pine nut material, a brazil nut material, or a pecan material.

Embodiment 59. The method of any one of embodiments 46-58, comprising pressing or milling the nut material prior to contacting the nut material with the supercritical fluid.

Embodiment 60. The method of any one of embodiments 46-59, further comprising characterizing one or more nut protein allergens in the defatted nut material.

Embodiment 61. The method of embodiment 60, wherein the one or more nut protein allergens are characterized using an enzyme linked immunosorbent assay (ELISA), reversed-phase high performance liquid chromatography (HPLC), size-exclusion chromatography (SEC), mass spectrometry, liquid chromatography-mass spectrometry (LC-MS), liquid chromatography-mass spectrometry/mass spectrometry (LC-MS/MS), or an immunoblot.

Embodiment 62. The method of any one of embodiments 46-61, further comprising separating larger particles in the nut flour from smaller nut particles in the nut flour, and retaining the smaller nut particles.

Embodiment 63. The method of embodiment 62, wherein the larger particles are separated from the smaller nut particles using one or more sieves.

Embodiment 64. The method of any one of embodiments 46-63, wherein contacting the nut material with the supercritical fluid comprises flowing the supercritical fluid through the nut material.

Embodiment 65. A method of treating a tree nut allergy in a subject, comprising orally administering to the subject an effective amount of a pharmaceutical composition comprising defatted tree nut flour, wherein the effective amount of the pharmaceutical composition comprises about 0.1 mg to about 2000 mg tree nut protein.

Embodiment 66. The method of embodiment 65, wherein the effective amount of the pharmaceutical composition comprises about 0.5 mg to about 1000 mg tree nut protein.

Embodiment 67. The method of embodiment 65 or 66, wherein at least 50% of the defatted tree nut flour by weight passes through a 250 µm sieve.

Embodiment 68. The method of any one of embodiments 65-67, wherein approximately all of the defatted tree nut flour passes through a 1 mm sieve.

Embodiment 69. The method of any one of embodiments 65-68, wherein approximately all of the defatted tree nut flour passes through a 250 µm sieve.

Embodiment 70. The method of any one of embodiments 65-69, wherein approximately all of the defatted tree nut flour passes through a 149 µm sieve.

Embodiment 71. The method of any one of embodiments 65-70, wherein approximately all of the defatted tree nut flour passes through a 74 µm sieve.

Embodiment 72. The method of any one of embodiments 65-71, wherein the defatted tree nut flour has an oil content of less than about 12% by weight.

Embodiment 73. The method of any one of embodiments 65-72, wherein the defatted tree nut flour has an oil content of less than about 6% by weight.

Embodiment 74. The method of any one of embodiments 65-73, wherein the tree nut is walnut, almond, pecan, cashew, hazelnut, pine nut, brazil nut, or pistachio.

Embodiment 75. A method of treating a nut allergy in a subject, comprising orally administering to the subject an effective amount of a pharmaceutical composition comprising defatted nut flour, wherein the defatted nut flour is produced according to a method comprising contacting a nut material with a supercritical fluid, thereby reducing the oil content of the nut material to form the defatted nut flour.

Embodiment 76. The method of embodiment 75, wherein the effective amount of the pharmaceutical composition comprises about 0.1 mg to about 2000 mg nut protein.

Embodiment 77. The method of embodiment 75 or 76, wherein the effective amount of the pharmaceutical composition comprises about 0.5 mg to about 1000 mg nut protein.

Embodiment 78. The method of any one of embodiments 75-77, wherein the method of producing the defatted nut flour further comprises milling the defatted nut flour.

Embodiment 79. The method of any one of embodiments 75-78, wherein the method of producing the defatted nut flour further comprises pressing or milling the nut material prior to contacting the nut material with the supercritical fluid.

Embodiment 80. The method of any one of embodiments 75-79, wherein at least 50% of the defatted nut flour by weight passes through a 250 µm sieve.

Embodiment 81. The method of any one of embodiments 75-80, wherein approximately all of the defatted nut flour passes through a 1 mm sieve.

Embodiment 82. The method of any one of embodiments 75-81, wherein approximately all of the defatted nut flour passes through a 250 µm sieve.

Embodiment 83. The method of any one of embodiments 75-82, wherein approximately all of the defatted nut flour passes through a 149 µm sieve.

Embodiment 84. The method of any one of embodiments 75-83, wherein approximately all of the defatted tree nut flour passes through a 74 µm sieve.

Embodiment 85. The method of any one of embodiments 75-84, wherein the defatted nut flour has an oil content of less than about 12% by weight.

Embodiment 86. The method of any one of embodiments 75-85, wherein the defatted nut flour has an oil content of less than about 6% by weight.

Embodiment 87. The method of any one of embodiments 75-86, wherein the defatted nut flour is a peanut flour.

Embodiment 88. The method of any one of embodiments 75-86, wherein the defatted nut flour is a tree nut flour.

Embodiment 89. The method of embodiment 88, wherein the tree nut is walnut, almond, pecan, cashew, hazelnut, pine nut, brazil nut, or pistachio.

Embodiment 90. The method of any one of embodiments 75-89, wherein the supercritical fluid is supercritical carbon dioxide.

Embodiment 91. The method of any one of embodiments 65-90, wherein the pharmaceutical composition comprises a carrier material.

Embodiment 92. The method of embodiment 91, wherein the carrier material comprises one or more diluents, glidants, or lubricants.

Embodiment 93. The method of any one of embodiments 65-92, wherein the pharmaceutical composition is administered daily.

Embodiment 94. The method of embodiment 93, wherein the same effective amount of the pharmaceutical composition is administered daily for at least one week.

Embodiment 95. The method of embodiment 65-94, wherein the effective amount of the pharmaceutical composition is periodically increased.

Embodiment 96. The method of any one of embodiments 65-95, wherein the effective amount of the pharmaceutical composition is adjusted to a different effective amount of the pharmaceutical composition after a period of at least one week, wherein the different effective amount of the pharmaceutical composition comprises about 0.1 mg to about 2000 mg tree nut protein.

Embodiment 97. The method of any one of embodiments 65-96, wherein the effective amount of the pharmaceutical composition is adjusted to a different effective amount of the pharmaceutical composition after a period of at least one week, wherein the different effective amount of the pharmaceutical composition comprises about 0.5 mg to about 1000 mg tree nut protein.

Embodiment 98. The method of any one of embodiments 65-97, wherein the pharmaceutical composition is administered daily for at least one month.

Embodiment 99. The method of any one of embodiments 65-98, wherein the subject is a human.

Embodiment 100. A method of manufacturing an ultra-low fat nut material comprising contacting a nut material having an initial oil content with a supercritical fluid to provide a defatted nut material having a reduced oil content, wherein the defatted nut material is allergenic.

Embodiment 101. The method of embodiment 100 wherein the defatted nut material has an oil content between 0.1% and 12%.

Embodiment 102. The method of embodiment 100 wherein the supercritical fluid is CO₂.

Embodiment 103. The method of any of embodiments 100-102 wherein the nut material is one of peanut or tree nut.

Embodiment 104. The method of any one of embodiments 100-103 wherein the nut material is tree nut comprising walnut, cashew, hazelnut, almond, pistachio, pine nut, brazil nut, or pecan.

Embodiment 105. The method of embodiment 100 or embodiment 102 wherein the nut material is course ground prior to contact with the supercritical fluid.

Embodiment 106. The method of embodiment 100 wherein the nut material is peanut, and wherein the reduced oil content is less than about 12% oil content, less than about 10% oil content, less than about 8% oil content, less than about 6% oil content, less than about 5% oil content, between about 3% oil content and about 7% oil content, between about 3% oil content and about 5% oil content, or about 4% oil content.

Embodiment 107. The method of embodiment 100 wherein the nut material is a tree nut, and wherein the reduced oil content is less than about 12% oil content, less than about 10% oil content, less than about 8% oil content, less than about 6% oil content, less than about 5% oil content, less than about 1% oil content, between about 0.1% oil content and about 7% oil content, between about 0.1% and 1% oil content, between about 1% and about 5% oil content, about 2% oil content or about 3% oil content.

Embodiment 108. A method of manufacturing a nut protein formulation, comprising: adding an amount of defatted nut material of embodiment 100 having characterized nut allergen proteins to one or more diluents, one or more glidants or one or more lubricants to form a powder; blending the powder; and encapsulating the blended powder in a capsule.

Embodiment 109. A method of treating tree nut allergy in a subject, comprising:
(a) providing a subject having one or more tree nut allergies;
(b) orally administering a pharmaceutical composition comprising ultra-low-fat tree nut flour, wherein the ultra-low-fat tree nut flour comprises a dose of 0.5 mg - 1000 mg tree nut protein and wherein the pharmaceutical composition is administered daily.

Embodiment 110. A method of treating tree nut allergy in a subject according to embodiment 109, wherein the dose of tree nut protein is about 3 mg, 6 mg, 12 mg, 20 mg, 40 mg, 80 mg, 120 mg, 160 mg, 200 mg, 240 mg or 300 mg.

Embodiment 111. A method of treating tree nut allergy in a subject according to embodiment 110, wherein the dose of tree nut protein is 3 mg.

Embodiment 112. A method of treating tree nut allergy in a subject according to embodiment 110, wherein the dose of tree nut protein is 6 mg.

Embodiment 113. A method of treating tree nut allergy in a subject according to embodiment 110, wherein the dose of tree nut protein is 12 mg.

Embodiment 114. A method of treating tree nut allergy in a subject according to embodiment 110, wherein the dose of tree nut protein is 20 mg.

Embodiment 115. A method of treating tree nut allergy in a subject according to embodiment 110, wherein the pharmaceutical composition is delivered daily for at least one month.

Embodiment 116. A method of treating tree nut allergy in a subject, comprising:
(a) providing a subject having one or more tree nut allergies;
(b) providing a pharmaceutical composition comprising ultra-low-fat tree nut flour having a dose of 0.5 mg - 1000 mg tree nut protein wherein the ultra-low-fat tree nut flour has been manufactured according to embodiment 100;
(c) orally administering said pharmaceutical composition to the subject on a daily basis for at least one month.

Embodiment 117. A method of treating tree nut allergy in a subject, comprising:
(a) providing a subject having one or more tree nut allergies;
(b) providing a pharmaceutical composition comprising ultra-low-fat tree nut flour having a dose of 0.5 mg - 1000 mg tree nut protein, wherein the ultra-low-fat tree nut flour has been manufactured according to embodiment 102;
(c) orally administering said pharmaceutical composition to the subject on a daily basis for at least one month.

Embodiment 118. A method of treating peanut allergy in a subject, comprising:
(a) providing a subject having a peanut allergy;
(b) orally administering a pharmaceutical composition comprising ultra-low-fat peanut flour, wherein the ultra-low-fat peanut flour comprises a dose of 0.5 mg - 1000 mg peanut protein and wherein the pharmaceutical composition is administered daily.

Embodiment 119. A method of treating peanut allergy in a subject according to embodiment 118, wherein the dose of peanut protein is 3 mg.

Embodiment 120. A method of treating peanut allergy in a subject according to embodiment 118, wherein the dose of peanut protein is 6 mg.

Embodiment 121. A method of treating peanut allergy in a subject according to embodiment 118, wherein the dose of peanut protein is 12 mg.

Embodiment 122. A method of treating peanut allergy in a subject according to embodiment 118, wherein the dose of peanut protein is 20 mg.

Embodiment 123. A method of treating peanut allergy in a subject according to embodiment 118, wherein the dose of peanut protein is 40 mg.

Embodiment 124. A method of treating peanut allergy in a subject according to embodiment 118, wherein the pharmaceutical composition is delivered daily for at least one month.

Embodiment 125. A method of treating peanut allergy in a subject, comprising:
(a) providing a subject having a peanut allergy;
(b) providing a pharmaceutical composition comprising ultra-low-fat tree peanut flour having a dose of 0.5 mg - 1000 mg peanut protein, wherein the ultra-low-fat nut flour is ultra-low-fat peanut flour and wherein said ultra-low-fat peanut flour has been manufactured according to embodiment 100;
(c) orally administering said pharmaceutical composition to the subject on a daily basis for at least one month.

Embodiment 126. A method of treating peanut allergy in a subject, comprising:
(a) providing a subject having a peanut allergy;
(b) providing a pharmaceutical composition comprising ultra-low-fat nut flour having a dose of 0.5 mg - 1000 mg peanut protein, wherein the ultra-low-fat peanut flour has been manufactured according to embodiment 102;
(c) orally administering said pharmaceutical composition to the subject on a daily basis for at least one month.

### VIII. EXAMPLES

The present technology may be better understood by reference to the following non-limiting examples. The following examples are presented in order to more fully illustrate certain embodiments and should in no way be construed, however, as limiting the broad scope of the present technology. While certain embodiments of the present technology have been shown and described herein, it will be obvious that such embodiments are provided by way of example only. Numerous variations, changes, and substitutions may occur to those skilled in the art without departing from the embodiments; it should be understood that various alternatives to the embodiments described herein may be employed in practicing the methods described herein.

### Example 1

This example describes oil extraction of dry roasted peanuts with supercritical CO₂ (sCO₂) for producing an ultra-low fat, stable, and hyper-allergenic peanut flour.

In this example, whole dry roasted peanuts (“peanuts”) were used as the feed stock for analyses and sCO₂ extractions. In a first analytical step, peanuts were separated and each of three portions were ground into coarse, medium and fine grind samples. Fractions of the “course” and “fine” ground portions were subsequently analyzed by hexane Soxhlet extraction to determine initial oil content of the peanuts and the volatiles content of the peanuts. As shown in Table 2 below, both coarse and fine grind fractions yielded an oil content of slightly less than 53%. The small difference between the coarse and find grind fractions indicate that mass transfer would not be a significant issue.

TABLE 2

Soxhlet Analysis of Dry Roasted Peanuts

| | Coarse (%) | Fine (% ) |
|---|---|---|
| Oil | 52.7 | 52.8 |
| Oil + Volatiles | 54.3 | 55.8 |
| Volatiles (Moisture, by difference) | 1.7 | 3.0 |

Oil extraction from the ground peanut material proceeded in two phases: the first phase designed to remove about ½ of the oil from whole or coarse ground peanuts at mild conditions, and the second phase designed to remove the remaining extractable oil from ground peanuts.

Whole peanuts were loaded into an extractor, and relatively low pressure (e.g., approximately 5,000 psi), low temperature (e.g., approximately 35° C.), and carbon dioxide (CO₂) was used for the extraction. At these initial conditions, about 6% of the feed material oil content was extracted (with approximately 47% remaining). The temperature was increased to 40° C. and an additional 3% oil content was extracted. The pressure was then increased to 9,000 psi, and an additional 2% oil content was extracted. The pressure was then held constant and the temperature was increased to 50° C. This step yielded an additional 17% oil extraction, for a total of 28% of the feed material oil content extracted.

The peanuts, still primarily intact, were removed from the extractor, ground and reloaded into the extractor. The ground peanuts, with about ½ of the oil already removed, were extracted at 9,000 psi and between 40 - 50° C. until 51.6% of the initial feed mass had been collected as oil extract. Table 3 shows sCO₂ extraction steps of the dry roasted peanuts of this example.

TABLE 3

Summary of sCO2 Dry Roasted Peanut Extraction Conditions

| Feed Material | Time | Pressure | Temperature | Extract as Fraction of Feed | Total Extract as Fraction of Feed |
|---|---|---|---|---|---|
| | (hr) | (psi) | (°C) | | |
| Whole peanuts | 1.3 | 4,500 -6,000 | 17-36 | 6% | 6% |
| | 3.8 | 4,900-5,300 | 37-42 | 3% | 9% |
| | 1.25 | 9,000-9,300 | 39-44 | 2% | 11% |
| | 1.25 | 8,100-9,500 | 46-52 | 17% | 28% |
| Grind peanuts and refill extractor | 5.2 | 6,000-9,900 | 28-53 | 24% | 52% |

The ground extracted peanut solids were removed from the extractor and weighed. Of the 109 g of peanut solids, 7 g were used for hexane Soxhlet extraction to determine residual oil content, and 102 g were sieved to determine particle size distribution.

As shown in Table 4, the residual oil content of the ground extracted peanut solids was 4.1%, as analyzed by hexane Soxhlet extraction.

TABLE 4

Soxhlet Analysis of Extracted Dry Roasted Peanuts

| | |
|---|---|
| Oil | 4.1% |
| Oil + Volatiles | 5.9% |
| Volatiles (Moisture, by difference) | 1.8% |

Table 4 shows particle size analysis of the extracted peanut material. The sieved analysis indicated the material (“Extracted”) was coarse compared with comparable peanut flour product analysis, so the peanut solid coarse fraction was reground and sieved (“Ground Peanuts”). The Ground Peanuts coarse fraction was ground again (“Re-Ground Peanuts”) and sieved. The resultant particle size analysis matched the comparable peanut flour products particle size (Table 5).

TABLE 5

Sieve Analysis of Extracted Dry Roasted Peanuts on Sieve Trays

| Sieve Tray | Extracted Peanuts | Ground Peanuts | Re-Ground Peanuts | Comparable Product | Difference |
|---|---|---|---|---|---|
| | % | % | % | % | % |
| 20 | 45.4 | 29.5 | 23.7 | 13.7 | 10.0 |
| 40 | 19.4 | 28.9 | 32.5 | 37.4 | -4.9 |
| 60 | 16.5 | 15.6 | 17.5 | 13.2 | 4.3 |
| 80 | 10.3 | 5.8 | 5.7 | 5.8 | -0.1 |
| 100 | 4.9 | 8.3 | 7.6 | | 7.6 |
| 200 | | | | 24.7 | -24.7 |
| pan | 3.6 | 12.0 | 13.0 | 5.2 | 7.7 |
| Total | 100.0 | 100.0 | 100.0 | 100.0 | 0.0 |

### Example 2

This example describes the characterization of the sCO₂ extracted peanut flour of Example 1. In particular, this example describes protein characterization of the resultant sCO₂ extracted peanut (“SEP”) flour (derived from roasted peanuts) as compared to petroleum ether extracted peanut protein (“CPP”) (derived from raw peanuts) and peanut flour defatted by pressing (derived from roasted peanuts) obtained from Golden Peanut Company (“GPC”) of Alpharetta Georgia, U.S.A. (lot No. 114FA21613).

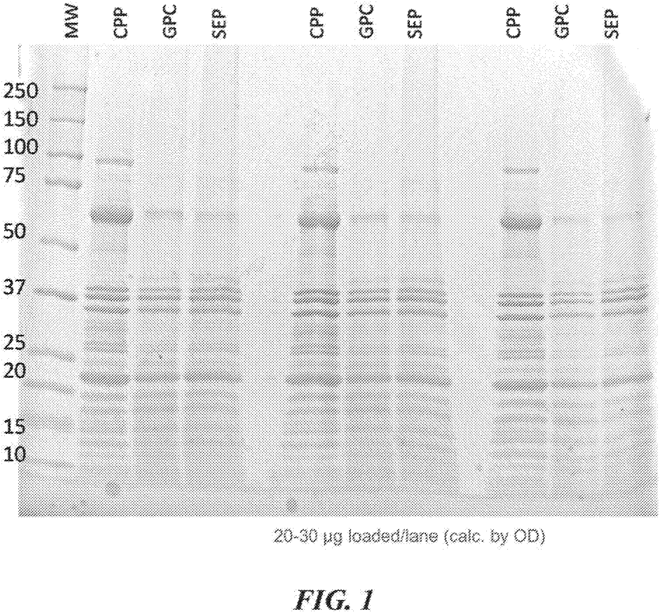

The ultra-low fat peanut flour of Example 1 having 4.1% residual oil was analyzed for total protein content using SDS polyacrylamide gel electrophoresis (SDS-PAGE) and stained with Coomassie blue. FIG. 1 shows the result SDS-PAGE separated proteins from SEP, GPC, and CPP (laboratory standard for peanut protein characterization). As shown, the total separated protein content looks comparable for the various peanut flour products.

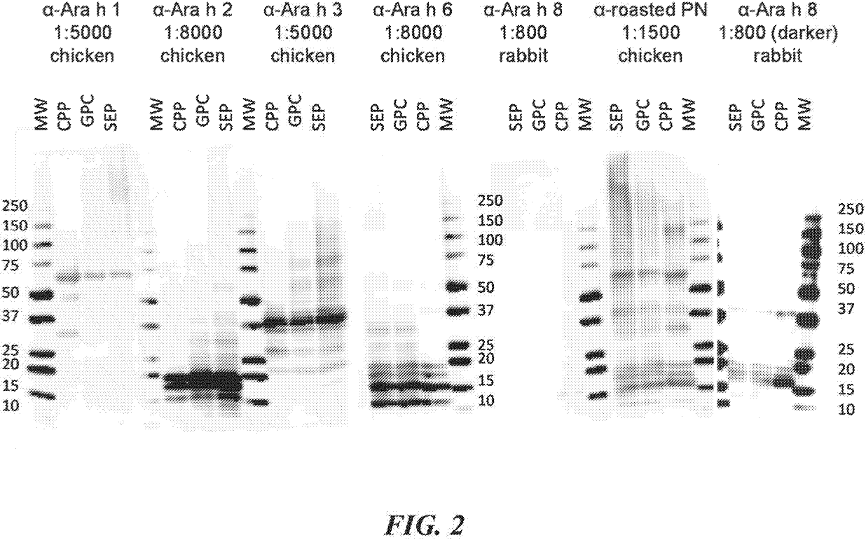

FIG. 2 shows immunoblot analysis of SDS-PAGE separated proteins from SEP, CPP, and GPC. SDS-PAGE separated proteins were transferred to polyvinylidene difluoride (PVDF) membranes and processed for immunoblotting with chicken or rabbit antisera to peanut allergens (e.g., Ara h 1, Ara h 2, Ara h 3, Ara-h 6, Ara-h 8, and peanut). Following reaction to primary antisera, the membranes were incubated with anti-chicken-HRP or anti-rabbit-HRP. As shown in FIG. 2, the SEP flour immunoblots were comparable to CPP and GPC blots.

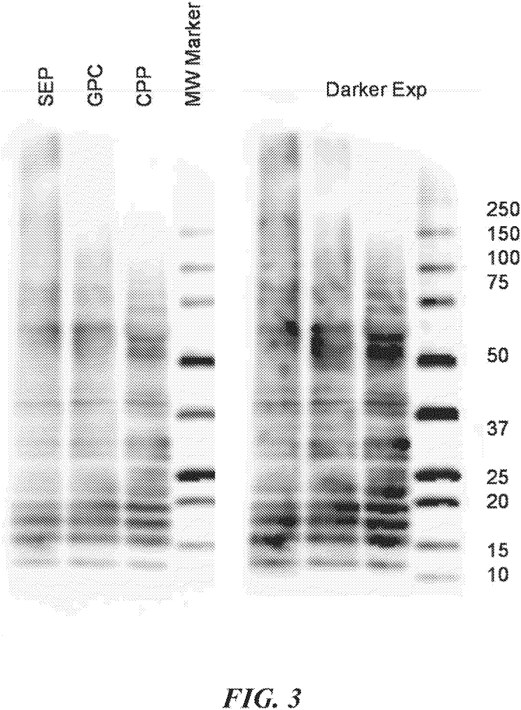

FIG. 3 shows immunoblot analysis of SDS-PAGE separated proteins from SEP, CPP, and GPC using pooled patient antisera. As shown, the patient serum reacted comparably with proteins from all the flours.

Reverse phase high performance liquid chromatography (RP-HPLC) was used to physically separate peanut protein allergens (e.g., Ara h 1, Ara h 2, Ara-h 6) as described in U.S. Pat. No. 9,198,869 which is incorporated herein by reference in its entirety. Generally, proteins from peanut flour must be extracted with an aqueous buffer prior to RP-HPLC analysis. A single stage extraction procedure was used with Tris buffer at pH 8.2, Flour samples were prepared at 100 mg/mL and extracted at 60° C. for 3 hours. Following centrifugation and filtration, the final neat filtrate was directly analyzed by HPLC using a wide pore 300 A silica column (Phenomenex® Jupiter C-4, 5 µm, 4.6 × 150 mm) with a bonded butyl stationary phase. Detection was accomplished with a UV detector at 214 nm.

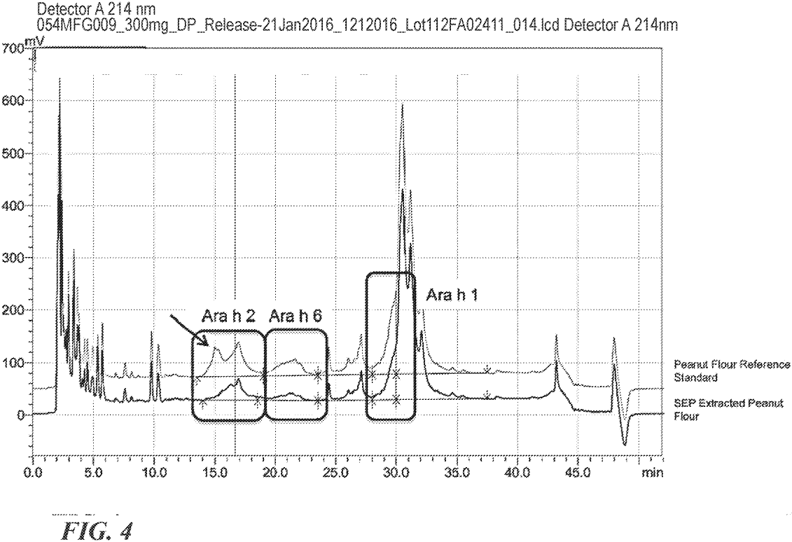

Peanut allergen characterization of SEP flour was determined by comparing the retention times and peak patterns of the SEP flour with a peanut flour reference standard. FIG. 4 is a chromatograph overlay of RP-HPLC separated proteins in accordance with aspects of the present technology. The principle Ara h protein peaks, in some instances, may not resolve as discrete entities, but rather may appear as ensembles of many similar proteins. Thus, the Ara h 1, Ara h 2 and Ara h 6 allergens may appear as clusters of peaks within a retention time region (FIG. 4). Accordingly, the relative amount of a particular Ara h protein is then determined as the percentage of the total area within a defined elution region. Chromatographic resolution of the various regions is assessed, and the method may be useful for comparison of subtle differences in these regional patterns for different lots and sources of peanut flour proteins, and stability of the formulation.

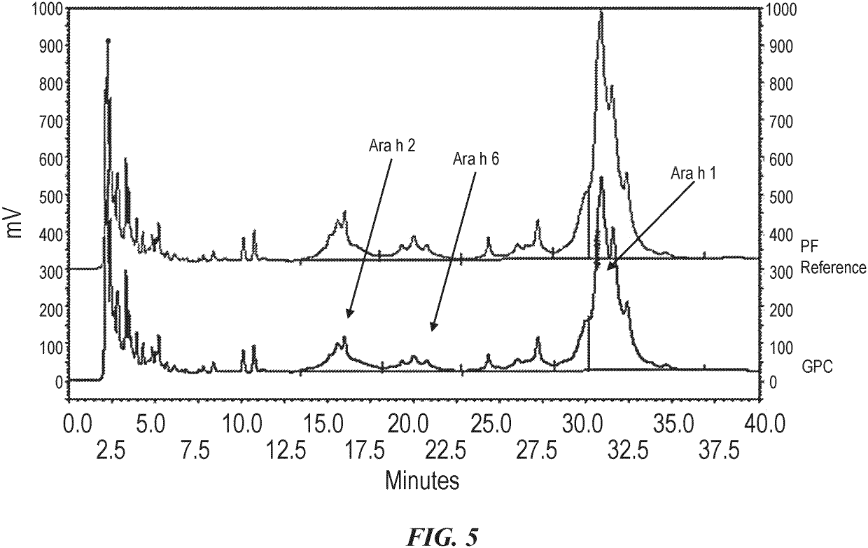

A chromatograph at 214 nm shown in FIG. 4 compares the SEP flour extract (bottom line) with a profile from a reference peanut flour sample having Ara h 1, Ara h 2 and Ara h 6 proteins (upper line). As shown in FIG. 4, the Ara h 1, Ara h 2 and Ara h 6 protein peaks are all present in the SEP flour chromatograph; however, some differences between the SEP flour chromatograph and the reference standard are seen in FIG. 4. With reference to the cluster of Ara h 2 peaks, the SEP flour chromatograph is absent the 15-minute peak (see arrow on reference standard). With reference to the cluster of Ara h 1 peaks, the leading shoulder of the first peak is smaller in the SEP flour chromatograph. Such differences in RP-HPLC with chromatograph analysis of Ara h 2 peaks, have also been seen with other peanut flour lots from GPC. FIG. 5 is a chromatograph overlay of RP-HPLC separated proteins from GPC peanut flour and a peanut flour reference standard in accordance with aspects of the present technology. As shown, the GPC peanut flour chromatograph (FIG. 5) has a similar profile to the SEP flour chromatograph (FIG. 4) at the cluster of Ara h 2 peaks. Accordingly, the variation between the peak clusters seen in FIG. 4 between SEP flour and the reference standard are considered to be within normal tolerance for oral immunotherapy formulations.

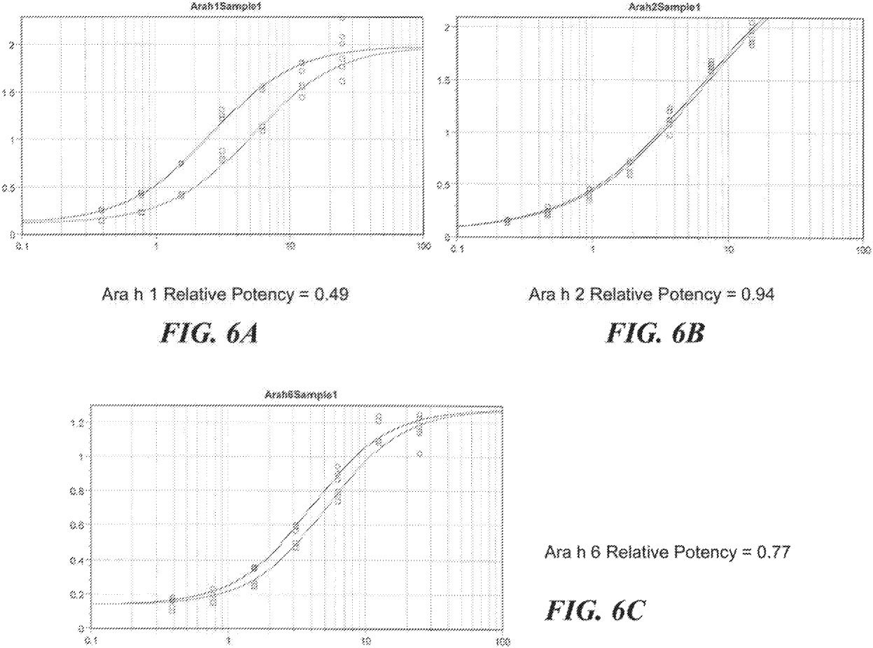

Additional characterization of the protein allergens was performed using Enzyme Linked Immunosorbent Assays (ELISA) with Ara h 1, Ara h 2 and Ara h 6 antibodies. FIGS. 6A-6C show detection of Ara h 1 (FIG. 6A), Ara h 2 (FIG. 6B), and Ara h 6 (FIG. 6C) in SEP flour (squares) and a reference standard peanut flour (circles). The extracted samples were normalized against each other for total protein content as determined by absorbance at 280 nm. With respect to the SEP flour, the relative potency of Ara h 2 and Ara h 6 are very consistent with the reference standard. The relative potency of Ara h 1 is below the reference standard, but extremely close to the predefined acceptable range, and is therefore considered therapeutically relevant.

Table 6 summarizes the protein characterization tests of SEP flour. As shown below, protein characterization of SEP flour conforms to the reference sample peanut flour with the exception of the Ara h 1 ELISA curve, and a slightly higher protein content.

TABLE 6

Protein Characterization of SEP Flour

| Test | Limits | SEP | Reference Sample |
|---|---|---|---|
| Loss on Drying | ≤ 9.0% | 3.2% | 3.1% |
| Protein Assay via Dumatherm | 46 - 52% (% wt/wt) | 54%(56% corrected for moisture) | 50% |
| HPLC | | | HPLC |
| Ara h 1 % Area | 5.0 - 13.0 | 9.9 | 9.5 |
| Ara h 2 % Area | 6.0 - 18.0 | 9.2 | 11.5 |
| Ara h 6 % Area | 3.0 - 9.0 | 4.0 | 5.3 |
| Ratio: Ara h 2/Ara h 6 | 1.3 - 2.7 | 2.3 | 2.2 |
| ELISA | | | ELISA |
| Ara h 1 | 0.5 -2.0 | 0.49 | 0.9 |
| Ara h 2 | 0.5 -2.0 | 0.94 | 1.1 |
| Ara h 6 | 0.5 -2.0 | 0.77 | 0.9 |

¹ Lot 115FA21014 Container 1
² Lot 115FA21014

Supercritical CO₂ extraction of roasted peanuts yielded milled flour with similar characteristics to milled flour from the Golden Peanut Company and crude peanut flour. Results shown in this Example demonstrate consistent reactivity patterns on total protein SDS-PAGE gels, immunoblots with specific anti-sera, and IgE immunoblots with pooled patient serum, and which were generally consistent to patterns from crude peanut protein (CPP) and a peanut flour from Golden Peanut Company (GPC).

### Example 3

This example describes oil extraction of dry roasted walnuts with sCO₂ for producing an ultra-low fat, stable and hyper-allergenic walnut flour.

In this example, whole dry roasted walnuts were used as the feedstock for analyses and sCO₂ extractions. In a first analytical step, dry roasted walnuts were course ground and a fraction was subsequently analyzed by hexane Soxhlet extraction to determine initial oil content and the volatiles content of the dry roasted walnuts. As shown in Table 7 below, the coarse ground dry roasted walnuts had an oil content of 70.7% and volatiles content of 0.9%.

TABLE 7

Soxhlet Analysis of Dry Roasted Walnuts

| | Dry Roasted (%) Coarse Grind |
|---|---|
| Oil | 70.7 |
| Oil + Volatiles | 71.6 |
| Volatiles (Moisture, by difference) | 0.9 |

Oil extraction from the ground dry roasted walnut feedstock proceeded in two phases: the first phase designed to remove about ½ of the oil from whole or coarse ground dry roasted walnuts at mild conditions, and the second phase that includes further grinding the dry roasted walnuts to sieve through 18 mesh and then to remove the remaining extractable oil from the finely ground dry roasted walnuts.

Coarsely ground dry roasted walnuts were separated into two batches of 231.84 grams and 232.31 grams, respectively. These batches were extracted with CO₂ at pressures from about 4,000 psi to about 7,000 psi and temperatures from about 30° C. - 50° C. The oil extract collected was 52.3% and 53.1% of the feed mass for the two batches; and the solid residue collected was 103 and 109 grams, respectively.

The solids from the first two batches were combined and sieved through an 18 mesh screen, and the residual coarse particles were ground in a Waring blender, sieved through 18 mesh, and this was repeated until all particles passed through 18 mesh. This material, with an estimated residual oil content of 37.7%, was extracted in two batches of 125.98 g and 78.72 g, respectively. The first batch (125.98 g) was extracted at pressures from about 4.000 psi to about 10,000 psi, and temperatures from about 25° C. - 45° C. The second batch (78.72 g) was extracted at pressures from about 6,000 psi to 8,000 psi, and temperatures from about 40° C. - 50° C. The extract collected was 35.7% and 40.3% of the feed mass; and the extracted flour collected from the two batches yielded 77.45 g and 45.95 g, respectively. Table 8 shows sCO₂ extraction steps of the dry roasted walnuts of this example.

TABLE 8

sCO₂ Extraction of Dry Roasted Walnuts

| Feed Material | Time | Pressure | Temperature | Flour Yield | Extract as Fraction of Feed |
|---|---|---|---|---|---|
| | (hr) | (psi) | (°C) | (g) | (%) |
| Dry Roasted Walnuts, Coarse Grind | 4 | 4,800-7,400 | 40-50 | 102.65 | 52.3 |
| Dry Roasted Walnuts, Coarse Grind | 3 | 6,800-7,200 | 32-49 | 105.79 | 53.1 |
| Dry Roasted Walnuts, Fine Grind, Re-extracted | 4 | 4,600-9,400 | 25-46 | 77.45 | 35.7 |
| Dry Roasted Walnuts, Fine Grind, Re-extracted | 2 | 6,200-8,500 | 40-50 | 45.95 | 40.3 |

The ground, extracted dry roasted walnut flour was combined. Hexane Soxhlet extraction on a fraction (8.12 g) was used to assess residual oil content in the flour. The remaining 123.4 g of flour was sieved. As shown in Table 9, the residual oil content of the ground extracted dry roasted walnut solids was 1.6%, as analyzed by hexane Soxhlet extraction. The volatiles in the solids, by difference of solids mass lost and oil collected in the Soxhlet extraction was 2.6%.

TABLE 9

Soxhlet Analysis of sCO₂ Extracted Dry Roasted Walnuts

| | Dry Roasted |
|---|---|
| | Coarse Grind |
| | (%) |
| Oil | 1.6 |
| Oil + Volatiles | 4.2 |
| Volatiles (by difference) | 2.6 |

Table 10 shows particle size analysis of the extracted dry roasted walnut flour.

TABLE 10

Sieve Analysis of sCO₂ Extracted Dry Roasted Walnuts Mass Fraction of Material on each Sieve Tray

| Sieve Tray | Extracted Dry Roasted Walnuts |
|---|---|
| | (%) |
| 20 | 3.9% |
| 40 | 23.9% |
| 60 | 20.7% |
| 80 | 14.5% |
| 100 | 4.00% |
| pan | 32.9% |
| Total | 100.0% |

### Example 4

This example describes oil extraction of raw walnuts with sCO₂ for producing an ultra-low fat, stable and hyper-allergenic walnut flour.

In this example, whole raw walnuts were used as the feedstock for analyses and sCO₂ extractions. In a first analytical step, raw walnuts were course ground and a fraction was subsequently analyzed by hexane Soxhlet extraction to determine initial oil content and the volatiles (presumably moisture) content of the dry roasted walnuts. As shown in Table 11 below, the coarse ground raw walnuts had an oil content of 69.9% and volatiles content of 2.3%.

TABLE 11

Soxhlet Analysis of Raw Walnuts

| | Raw (%) Coarse Grind |
|---|---|
| Oil | 69.9 |
| Oil + Volatiles | 72.2 |
| Volatiles (Moisture, by difference) | 2.3 |

Oil extraction from the ground raw walnut feedstock proceeded in two phases: the first phase designed to remove about ½ of the oil from coarse ground raw walnuts, and the second phase that includes further grinding the dry roasted walnuts to sieve through 18 mesh and then to remove the remaining extractable oil from the finely ground raw walnuts.

Coarsely ground raw walnuts were separated into two batches of 231.36 grams (batch 1) and 252.81 grams (batch 2), respectively. These batches were extracted with sCO₂. The first batch was extracted at a pressure of about 7,000 psi and temperatures from about 35° C. - 50° C., and the second batch was extracted at a pressure from about 6,000 psi to about 9,000 psi and temperatures from about 20° C. - 56° C. The oil extract collected was 52.2% and 49.5% of the feed mass for the two batches; and the solid residue collected was 103 and 114 grams, respectively.

The solids from the first two batches were combined and sieved through an 18 mesh screen, and the residual coarse particles were ground in a Waring blender, sieved through 18 mesh, and this was repeated until all particles passed through 18 mesh. This material (104.74 g), with an estimated residual oil content of 42.4%, was extracted at pressures from about 6,000 psi to about 8,000 psi, and temperatures from about 37° C. - 47° C. The extract collected was 52.3 % of the feed mass; and the extracted flour collected produced 49 grams. Table 12 shows sCO₂ extraction steps of the raw walnuts of this example.

TABLE 12

sCO₂ Extraction of Raw Walnuts

| Feed Material | Time | Pressure | Temperature | Flour Yield | Extract as Fraction of Feed |
|---|---|---|---|---|---|
| | (hr) | (psi) | (°C) | (g) | (%) |
| Raw Walnuts, Coarse Grind | 2.5 | 7,000-7,300 | 35-50 | 103.14 | 52.2 |
| Raw Walnuts, Coarse Grind | 3.5 | 6,100-8,800 | 18-56 | 105.37 | 49.5 |
| Raw Walnuts, Fine Grind, Re-extracted | 1.5 | 6,700-7,500 | 37-47 | 49.08 | 52.3 |
| Raw Walnuts, Fines, Re-extracted | 2 | 8,300-10,400 | 42-48 | 62.70 | 15.9 |

The ground, extracted raw walnut flour was used to assess residual oil content in the flour. The remaining 123.4 g of flour was sieved. As shown in Table 13, the residual oil content of the ground extracted raw walnut solids was 1.6%, as analyzed by hexane Soxhlet extraction. The volatiles in the solids, by difference of solids mass lost and oil collected in the Soxhlet extraction was 2.6%.

TABLE 13

Soxhlet Analysis of sCO₂ Extracted Raw Walnuts

| | Raw |
|---|---|
| | Coarse Grind |
| | (%) |
| Oil | 1.6 |
| Oil + Volatiles | 4.2 |
| Volatiles (by difference) | 2.6 |

Table 14 shows particle size analysis of the extracted raw walnut flour.

TABLE 14

Sieve Analysis of sCO₂ Extracted Raw Walnuts Mass Fraction of Material on each Sieve Tray

| Sieve Tray | Extracted Raw Walnuts |
|---|---|
| | (%) |
| 20 | 1.9% |
| 40 | 33.1% |
| 60 | 20.4% |
| 80 | 8.5% |
| 100 | 5.5% |
| pan | 30.6% |
| Total | 100.0% |

### Example 5

This example describes the characterization of the sCO₂ extracted dry roasted and raw walnut flours of Examples 3 and 4. In particular, this example describes protein characterization of the resultant sCO₂ extracted dry roasted walnut (“sCO₂ dry roasted”) flour and the sCO₂ extracted raw walnut flour (“sCO₂ raw”) as compared to crude (extracted with pet ether) walnut protein (“Walnut”), defatted (by pressing) raw walnut flours obtained from Pearl Crop (“Pearl”) of Stockton, CA, U.S.A. (lot No. 365735), and defatted (by pressing) raw walnut flours obtained from La Tourangelle (“La Tourangelle”) of Berkeley, CA, U.S.A. (lot No. WMP-14516).

Initial differences in the walnut flour samples include texture and color: the sCO₂ extracted dry roasted and raw walnut flours were pale beige in color and had a “fluffy” consistency, while the walnut flours obtained from pressing were dark brown and had a dense consistency.

Total protein was extracted from sCO₂ dry roasted, sCO₂ raw, Pearl, and La Tourangelle samples with extraction buffer (50 mM phosphate buffer pH 7.5, and 6 M urea) with the following protocol:
1. Weigh 10 mg of flour in a 1.5 mL centrifuge tube
2. Add 1 mL of either phosphate or urea buffer
3. Heat for 1 hour at 60° C. in shaker
4. Add 5 uL of walnut flour extract to 96 well plate
5. Add 200 uL of Bradford assay reagent (Sigma Aldrich, product # B6919)
6. Measure absorbance at 595 nm

Bovine serum albumin was used as the protein standard to calibrate the assay over the concentration range of 25 to 2000 µg/mL. The walnut flour samples were further diluted by 3 or 6 fold so the protein levels were within the calibration range.

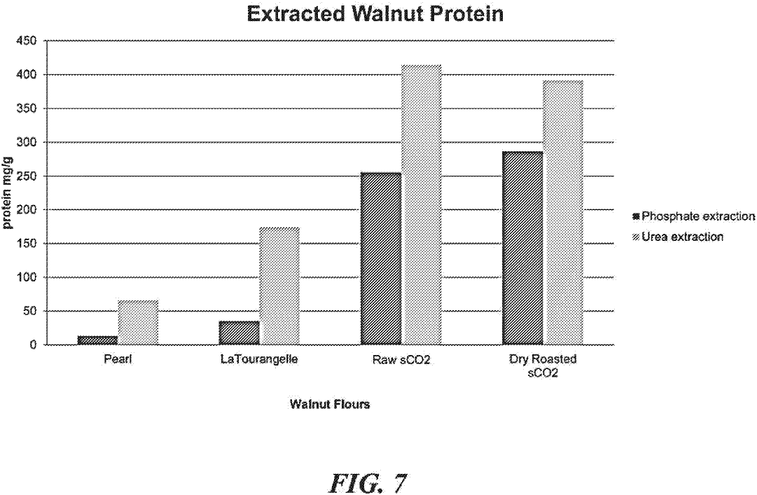

FIG. 7 is a graph illustrating total protein extracted from walnut flour samples. As shown in FIG. 7, sCO₂ extraction provided walnut flours with more extractable proteins.

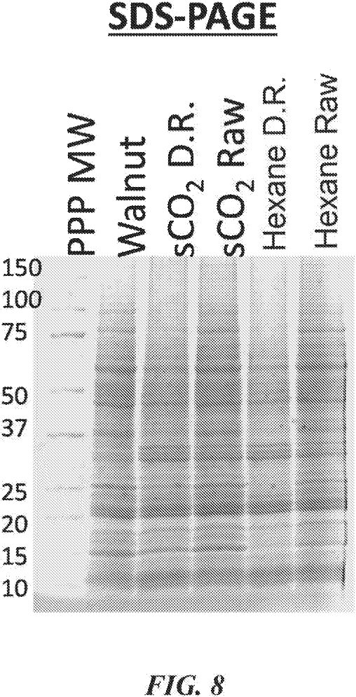

The total extracted proteins from pet ether crude walnut, sCO₂ dry roasted walnut, sCO₂ raw walnut, and hexane raw and dry roasted walnut flours were further characterized with respect to specific walnut allergens. The ultra-low fat dry roasted walnut flour of Example 3 having 1.6%% residual oil, and the ultra-low fat raw walnut flour of Example 4 having 2.7% residual oil were analyzed for total protein content using SDS-PAGE and stained with Coomassie blue. FIG. 8 shows the result of SDS-PAGE separated proteins from these flours. As shown, the total separated protein content looks comparable between the walnut flour products.

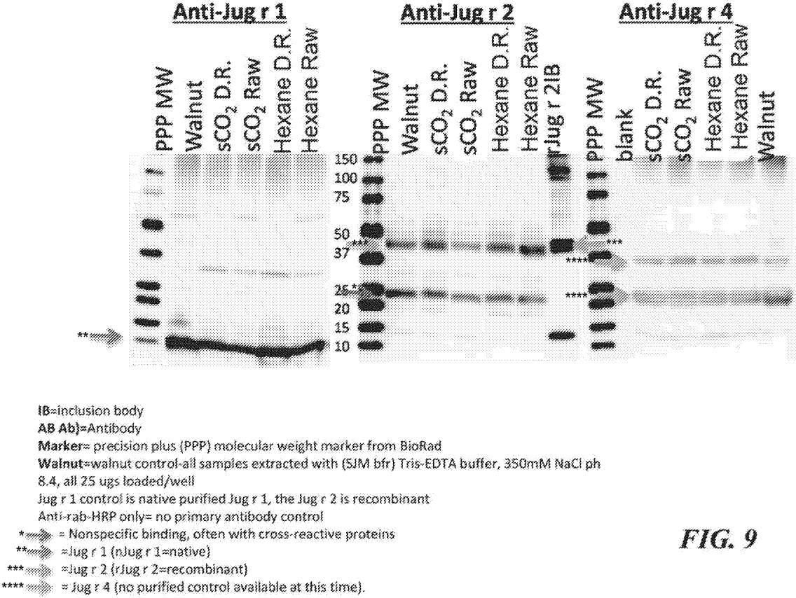

FIG. 9 shows immunoblot analysis of SDS-PAGE separated proteins from crude walnut, sCO₂ dry roasted walnut, sCO₂ raw walnut, and hexane walnut flours. SDS-PAGE separated proteins were transferred to polyvinylidene difluoride (PVDF) membranes and processed for immunoblotting with rabbit antisera to walnut proteins (e.g., Jug r 1, Jug r 2, and Jug r 4). Following reaction to primary antisera, the membranes were incubated with anti-rabbit-HRP. As shown in FIG. 9, the immunoblots from sCO₂ extracted flours (sCO₂ D.R. and sCO₂ Raw) were comparable to pet ether extracted walnut flour as well as proteins extracted from hexane extracted walnut flours in both protein size and quantity.

### Example 6

This example describes oil extraction of raw pecans with sCO₂ for producing an ultra-low fat, stable and hyper-allergenic pecan flour.

In this example, whole raw pecans were used as the feedstock for analyses and sCO₂ extractions. In a first analytical step, raw pecans were course ground and a fraction was subsequently analyzed by hexane Soxhlet extraction to determine initial oil content and the volatiles content of the raw pecans. As shown in Table 15 below, the coarse ground raw pecans had an oil content of 70.5% and volatiles content of 2.8%.

TABLE 15

Soxhlet Analysis of Raw Pecans

| | Raw (%) Coarse Grind |
|---|---|
| Oil | 70.5 |
| Oil + Volatiles | 73.3 |
| Volatiles (Moisture, by difference) | 2.8 |

The solids from the first two batches were kept separate and sieved through 18 mesh screen, and the coarse was ground in a Waring blender, sieved through 18 mesh, the coarse was ground again, and this was repeated until all passed through 18 mesh. This material, with a residual oil content of about 30%, was extracted in two batches - runs 3-1-2 (solids from run 8109-3-1) and 3-2-2 (solids from run 8109-2-1), with 92 and 87 g of feed, respectively. Run 3-1-2 was extracted at pressures from 7,200 to 7,600 psi, and temperatures from 33 - 61° C., however, the run was not completed in one day, but was shut down and restarted. Run 3-2-2 was extracted at pressures from 7,300 to 7,500 psi, and temperatures from 38 - 59° C. The extract collected was 32.6 and 25.8% of the feed mass; and the extracted flour collected from the two batches was 62 and 65 g, respectively. Extraction parameters are shown in Table 16. The extraction temperature was not as steady as the pressure, but after startup was typically 55 - 60° C. except for run 3-2-1, which started warm, but after shut down was restarted cold.

The ground extracted raw pecan flour was combined, then further ground in a Waring blender until it passed through 60 mesh. Of the 127 g, 6 g were used for hexane Soxhlet extraction, a small amount was lost during grinding and sieving, and the balance of the flour was prepared for shipping. The Soxhlet analysis of flour was 0.2 % oil content, meeting the residual oil target. The volatiles in the solids, by difference of solids mass lost and oil collected in the Soxhlet was 0.9%. See Table 17 for the Soxhlet analysis of the extracted raw pecan solids.

TABLE 16

sCO₂ Extraction of Raw Pecans

| Feed Material | Time | Pressure | Temperature | Flour Yield | Extract as Fraction of Feed |
|---|---|---|---|---|---|
| | (hr) | (psi) | (°C) | (g) | (%) |
| Raw Pecans, Coarse Grind 8109-3-1 | 3 | 6.800-7.400 | 49-61 | 92.34 | 55.3 |
| Raw Pecans, Coarse Grind 8109-3-2 | 3 | 7.000-7.400 | 40-60 | 86.79 | 61.8 |
| Raw Pecans, Fine Grind, Re-extracted 8109-3-1-2 | 2.5 | 7.200-7.600 | 33-61 | 62.05 | 32.6 |
| Raw Pecans, Fines, Re-extracted 8109-3-2-2 | 2 | 7.300-7.500 | 34-59 | 65.39 | 25.8 |

The ground, extracted raw pecan flour was used to assess residual oil content in the flour. As shown in Table 17, the residual oil content of the ground extracted raw pecan solids was 0.2%, as analyzed by hexane Soxhlet extraction. The volatiles in the solids, by difference of solids mass lost and oil collected in the Soxhlet extraction was 0.9%.

TABLE 17

Soxhlet Analysis of sCO₂ Extracted Pecan Flour

| | (%) |
|---|---|
| Oil | 0.2 |
| Oil + Volatiles | 1.1 |
| Volatiles (bv difference) | 0.9 |

### Example 7

This example describes blend formulation and processing of ultra-low fat dry roasted peanut flour extracted using sCO₂ (from Example 1).

Supercritical CO₂ extracted peanut flour (SEP) from Example 1 was sieved through a 20 mesh screen, and 30.75 g of screened material was transferred to a mixing vessel. AVICEL® PH-102 (63 g) was passed through a clean 20 mesh screen and transferred to the mixing vessel. Remaining AVICEL®PH-102 was transferred to a poly-bag containing 0.5 CAB-O-SIL®. SEP material and AVICEL®PH-102 were thoroughly mixed in the vessel and subsequently sieved through a 40 mesh screen, transferred to the mixing vessel, and blended for 20 minutes. Magnesium stearate was sieved through a 40 mesh screen, transferred to the mixing vessel and blended for 5 minutes. Table 18 shows the formulated blend as prepared for filling a 100 mg oral immunotherapy capsule.

TABLE 18

SEP Blend Formulation (100 mg Capsule)

| Material | %wt/wt | mg/capsule | g/batch |
|---|---|---|---|
| SEP flour | 16.67 | 184.5 (100) | 30.75 (16.67) |

| AVICEL^{®}PH-102 | 82.33 | 409.5 | 68.25 |
|---|---|---|---|
| CAB-O-SIL^{®} | 0.5 | 3.0 | 0.5 |
| Magnesium stearate | 0.5 | 3.0 | 0.5 |
| Total | 100 | 600 | 100 |

The resultant mixed material was a fine powder with suitable flow characteristics for current good manufacturing practices (CGMP) manufacturing. Table 19 shows relative density data and flow characteristic analysis for SEP peanut flour, SEP blended material, and commercially available peanut flour lots (Golden Peanut Company (GPC)). Table 20 references Carr’s Index and Hausner Ratio analysis.

TABLE 19

Density and Flow Characteristics of Peanut Flours

| Material | Bulk density (g/mL) | Tapped density (g/mL) | Carr’s Index (%) | Hausner Ratio |
|---|---|---|---|---|
| SEP Peanut flour | 0.249 | 0.383 | 35.0 | 1.54 |
| SEP 100 mg blend (L-0268-02) | 0.324 | 0.498 | 35.0 | 1.54 |
| Peanut flour (GPC lot 111FA36111 ) | 0.24 | 0.49 | 51.0 | 2.04 |
| Peanut flour (GPC lot 111FA36211) | 0.26 | 0.49 | 46.9 | 1.88 |
| Peanut flour (GPC lot 112FA02411) | 0.24 | 0.50 | 52.0 | 2.08 |

TABLE 20

Carr’s Index and Hausner Ratio Analysis

| Carr’s Index (%) | Flow Character | Hausner Ratio |
|---|---|---|
| <10 | Excellent | 1.00-1.11 |
| 11-15 | Good | 1.12-1.18 |
| 16-20 | Fair | 1.19-1.25 |
| 21-25 | Passable | 1.26-1.34 |
| 26-31 | Poor | 1.35-1.45 |
| 32-37 | Very poor | 1.46-1.59 |
| >38 | Very, very poor | >1.60 |

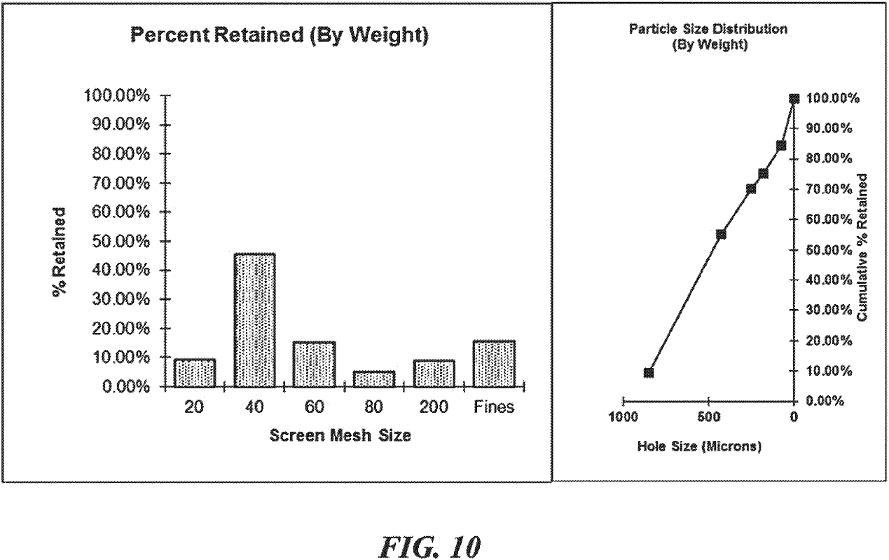

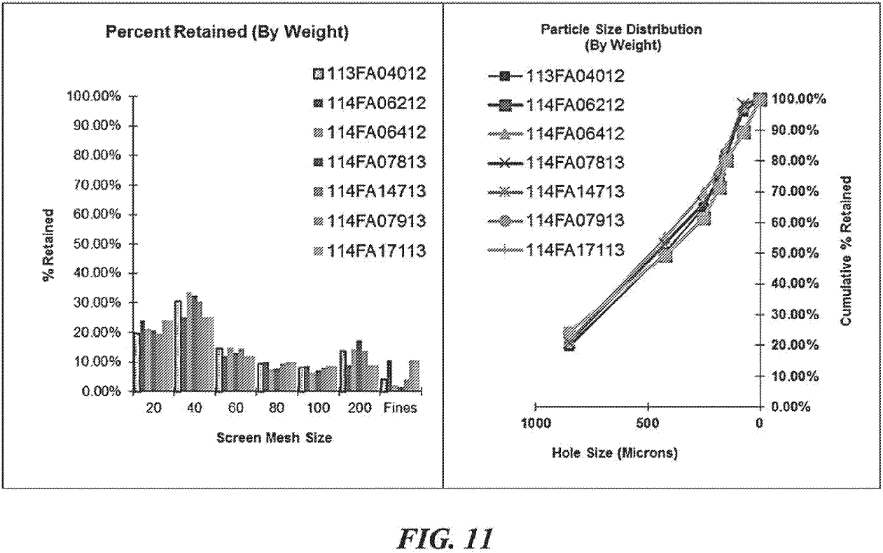

FIG. 10 shows particle size distribution for SEP peanut flour, and FIG. 11 shows particle size distribution for multiple commercially available peanut flour lots (GPC).

This example demonstrates that supercritical CO₂ extraction of oil from dry roasted peanuts can yield an ultra-low fat peanut flour suitable for formulation.

### Example 8

This example describes blend formulation and processing of ultra-low fat walnut flour flours extracted using sCO₂ (from Examples 3 and 4).

sCO₂ extracted walnut flour from Examples 3 and 4 were sieved through a 20 mesh screen, and the amount of screened material indicated in Tables 21 and 22, respectively, were transferred to mixing vessels. Approximately 5 g of AVICEL®PH-102 was transferred to a poly-bag containing 0.5 g CAB-O-SIL®. The remaining AVICEL®PH-102 was passed through a clean 20 mesh screen and transferred to the mixing vessel. The AVICEL®PH-102 and CAB-O-SIL® in the poly-bag were mixed thoroughly, passed through a 40 mesh screen, transferred to the mixing vessel, and blended for 20 minutes. Magnesium stearate was sieved through a 40 mesh screen, transferred to the mixing vessel and blended for 5 minutes. Tables 19 (a) and 19 (b) show the formulated blends as prepared for filling 600 mg oral immunotherapy capsules.

TABLE 21

Dry-Roasted Walnut Blend Formulation (600 mg Capsules)

| Material | %wt/wt | mg/capsule | g/batch |
|---|---|---|---|
| Dry roasted walnut flour (protein) | 33.51 (16.67) | 201.06 (100) | 33.51 (16.67) |
| AVICEL®PH-102 | 65.49 | 392.94 | 65.49 |
| CAB-O-SIL® | 0.5 | 3.0 | 0.5 |
| Magnesium stearate | 0.5 | 3.0 | 0.5 |
| Total | 100 | 600 | 100 |

TABLE 22

Raw-Walnut Blend Formulation (600 mg Capsules)

| Material | %wt/wt | mg/capsule | g/batch |
|---|---|---|---|
| Raw walnut flour (protein) | 34.53 (16.67) | 207.18 (100) | 34.53 (16.67) |
| AVICEL®PH-102 | 64.47 | 386.82 | 64.47 |
| CAB-O-SIL® | 0.5 | 3.0 | 0.5 |
| Magnesium stearate | 0.5 | 3.0 | 0.5 |
| Total | 100 | 600 | 100 |

The resultant mixed material was a fine powder with suitable flow characteristics for CGMP manufacturing. Table 23 shows relative density data and flow characteristic analysis for sCO₂ extracted walnut blend formulations set forth in Tables 19 (a) and 19 (b).

TABLE 23

Density and Flow Characteristics of sCO₂ Extracted Walnut Formulations

| Material | Bulk density (g/mL) | Tapped density (g/mL) | Carr’s Index (%) | Hausner Ratio |
|---|---|---|---|---|
| Dry Roasted Walnut Flour | 0.167 | 0.26 | 36.0 | 1.56 |
| Raw Walnut Flour | 0.165 | 0.26 | 36.0 | 1.58 |
| 100 mg blend (dry roasted walnut) | 0.281 | 0.426 | 34.0 | 1.52 |
| 100 mg blend (raw walnut) | 0.271 | 0.41 | 33.0 | 1.51 |

As shown in Table 23, the flow characteristics of the dry roasted walnut flour is very similar to that of the raw walnut flour.

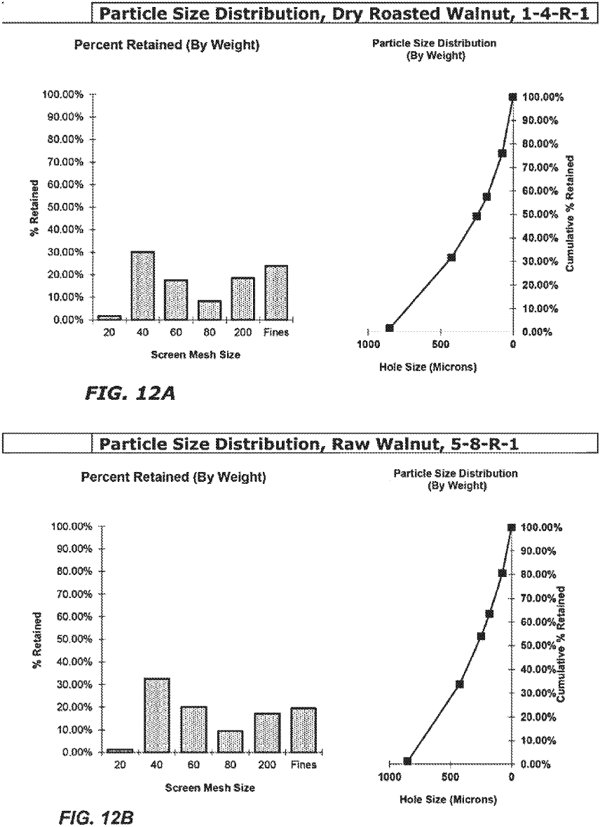

Also Particle Size Distribution (PSD) by Sieve Analysis was conducted using a 10 g sample of each walnut flour. For this test, the sample was pre-screened through a 20 mesh and a horizontal pulse unit replaced the 100 mesh screen. PSD data for the dry roasted walnut flour are shown in FIG. 12A and for raw walnut in FIG. 12B.

This example demonstrates that sCO₂ extraction of oil from both dry roasted and raw walnuts can yield an ultra-low fat walnut flour suitable for formulation.

### Example 9

This example describes blend formulation and processing of ultra-low fat pecan flour extracted using sCO₂ (from Example 6).

sCO₂ extracted pecan flour from Example 6 were sieved through a 20 mesh screen, and the amount of screened material indicated in Table 21 was transferred to mixing vessels. Approximately 5 g of AVICEL®PH-102 was transferred to a poly-bag containing 0.5 g CAB-O-SIL®. The remaining AVICEL®PH-102 was passed through a clean 20 mesh screen and transferred to the mixing vessel. The AVICEL®PH-102 and CAB-O-SIL® in the poly-bag were mixed thoroughly, passed through a 40 mesh screen, transferred to the mixing vessel, and blended for 20 minutes. Magnesium stearate was sieved through a 40 mesh screen, transferred to the mixing vessel and blended for 5 minutes. Table 24 shows the formulated blend as prepared for filling 600 mg oral immunotherapy capsules.

TABLE 24

Raw Pecan Blend Formulation (600 mg Capsules)

| Material | %wt/wt | mg/capsule | g/batch |
|---|---|---|---|
| Raw pecan flour (protein) | 40.29 (16.67) | 241.74 (100) | 40.29 (16.67) |
| AVICEL®PH-102 | 58.71 | 352.26 | 58.71 |
| CAB-O-SIL® | 0.5 | 3.0 | 0.5 |
| Magnesium stearate | 0.5 | 3.0 | 0.5 |
| Total | 100 | 600 | 100 |

The resultant mixed material was a fine powder with suitable flow characteristics for cGMP manufacturing. Table 25 shows relative density data and flow characteristic analysis for sCO₂ extracted pecan blend formulation set forth in Table 24.

TABLE 25

Density and Flow Characteristics of sCO₂ Extracted Pecan Formulations

| Material | Bulk density (g/mL) | Tapped density (g/mL) | Carr’s Index (%) | Hausner Ratio |
|---|---|---|---|---|
| Raw Pecan Flour | 0.156 | 0.294 | 46.9 | 1.88 |
| 100 mg blend (raw pecan) | 0.242 | 0.441 | 45 | 1.82 |

The flow characteristics of raw pecan flour were similar to that of raw pecan flour blend and acceptable for manufacturing applications.

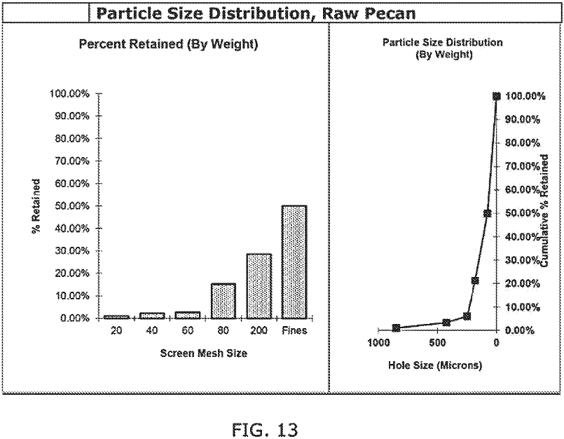

Particle Size Distribution (PSD) by Sieve Analysis was conducted using a 10 g sample of the pecan flour. For this test, the sample was pre-screened through a 20 mesh and a horizontal pulse unit replaced the 100 mesh screen. PSD data for the raw pecan flour are shown in FIG. 13.

This example demonstrates that sCO₂ extraction of oil from raw pecans can yield an ultra-low fat pecan flour suitable for formulation.

### Example 10

Walnut pieces were fed into a screw oil expeller and pressed to remove a first fraction of fast and to reduce the walnut particle size. Pressing of the walnut pieces reduced the fat content of the walnut material from about 70% to about 54%, as measured by hexane Soxhlet extraction.

Supercritical carbon dioxide was warmed to approximately 60° C. and pressurized to about 40 MPa, and allowed to flow through 6 kg of pressed walnut material for approximately 4 hours at a rate of approximately 1 kg/min. The residual fat content of the walnut flour was 4.2%, as measured by hexane Soxhlet extraction.

The defatted walnut flour was milled and passed through a series of sieves. The defatted walnut flour was milled easily, with approximately 96% of the walnut flour by weight passing through a 500 µm (35 mesh) sieve, and about 38% of the walnut flour by weight passing through a 149 µm (100 mesh) sieve.

### Example 11

Walnut flour samples as detailed below were prepared for RP-HPLC analysis by mixing the walnut flour with a high-salt Tris NaCl buffer (25 mM Tris-HCl, pH 7.5, 1.5 M NaCl, 3.5% (w/v) poly(vinylpolypyrrolidone) at a walnut flour concentration of 10 mg/mL, and incubated for 1 hour at 40° C. to extract walnut proteins. During the incubation period, the sample was vortex for 20-30 seconds every 10 minutes. After protein extraction, the samples were centrifuged at 6000 rpm for 10 minutes, and the supernate was used for RP-HPLC analysis.

RP-HPLC analysis was perfumed using an Agilent Poroshell 300SB-C18, 2.1 x 75 mm, 5 µm particles column, quipped with a Poroshell guard column. 2.5 µL of extracted protein was injected onto the column Mobile phases included 0.2% TFA in water (phase A) and 95% acetonitrile in water with 0.2% TFA (phase B). The mobile phase gradient began with 100% mobile phase A, and mobile phase B was increased to 30% from minute 0 to minute 10, and to 52% from minute 10 to minute 45. Flow rate was held constant at 0.3 mL/min, and column temperature was held at 55° C. Data was collected by UV at 210 nm.

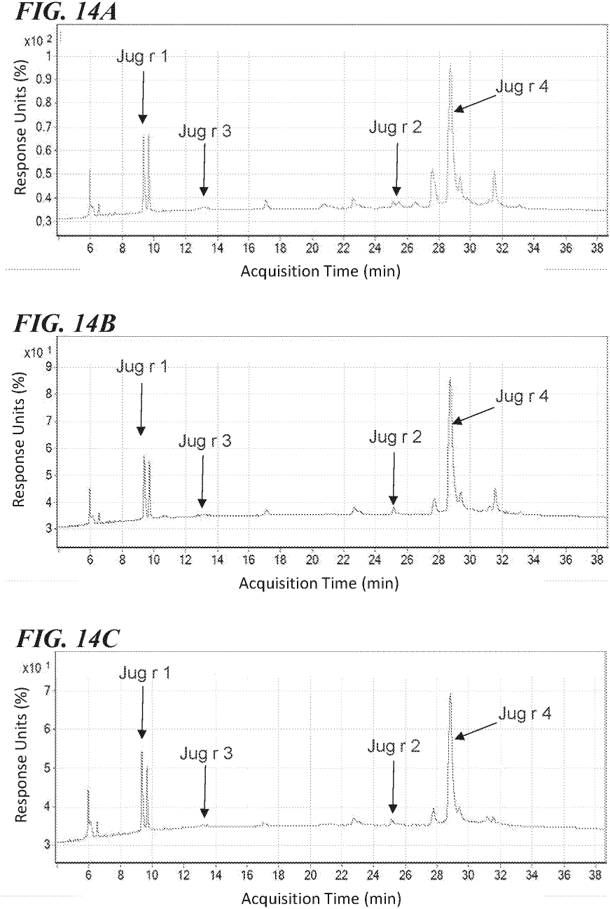

Sample 1: Steam-pasteurized walnut material was defatted by contacting the walnut material with supercritical carbon dioxide to form a defatted walnut flour. RP-HPLC analysis of the sample 1 is shown in FIG. 14A. Arrows in FIG. 14A indicate different walnut protein antigen peaks.

Sample 2: Steam-pasteurized walnut material was initially defatted using a screw oil expeller. Following an initial defatting using the oil expeller, the walnut material was further defatted by contacting the walnut material with supercritical carbon dioxide to form defatted walnut flour. RP-HPLC analysis of the sample 2 defatted walnut flour is shown in FIG. 14B. Arrows in FIG. 14B indicate different walnut protein antigen peaks.

Sample 3: A commercially available defatted walnut flour (Bio Planete, Lommatzsch, Germany). RP-HPLC analysis of the sample 3 is shown in FIG. 14C. Arrows in FIG. 14C indicate different walnut protein antigen peaks.

Walnut protein antigens eluted from the column with the following approximate retention times: Jug r 1: 9.6 minutes; Jug r 3: 13.2 minutes; Jug r 2: 25.1 minutes; Jug r 4: 28.7 minutes. Peak area for each protein antigen is shown in Table 26.

TABLE 26

Peak Area for RP-HPLC Analysis of Walnut Flours

| | Jug r 1 | Jug r 2 | Jug r 3 | Jug r4 |
|---|---|---|---|---|
| Sample 1 | 2059 | 400 | 239 | 7037 |
| Sample 2 | 1420 | 126 | 194 | 5330 |
| Sample 3 | 1130 | 96 | 124 | 3443 |

### Example 12

Jug r 1 and Jug r 2 protein antigens from walnut flour samples were analyzed by immunoblot. The analyzed samples are as follows: (1) steam-pasteurized walnut material defatted using supercritical carbon dioxide at about 45° C.; (2) steam-pasteurized walnut material defatted using supercritical carbon dioxide at about 75° C.; (3) commercially available walnut flour (Lot No. 08340148; Bio Planete, Lommatzsch, Germany); (4) commercially available walnut flour (Lot No. 07210081; Bio Planete, Lommatzsch, Germany); and (5) unpasteurized walnut material defatted using supercritical carbon dioxide at about 45° C.

To obtain walnut protein extracts, 0.4 grams of each walnut powder sample was mixed with borate saline buffer (10 mM H₃BO₃, 25 mM Na₂B₄O₇, 75 mM NaCl, pH 8.5) to a total volume of 10 mL. The samples were then chilled on ice before being sonicated and centrifuged. The supernatant was transferred and stored at either -20° C. or -80° C. Protein content was determined by UV absorption.

20 µg of protein extracted from each sample was analyzed using a 12% Bis-Tris SDS-PAGE gel before being transferred and stained using patient sera or purified antibody. As a control, isolated Jug r 1 or Jug r 2 was simultaneously analyzed. For the patient sera samples, sera was collected from five individuals with a diagnosed walnut allergy, pooled, and diluted 1:5 with phosphate buffered saline with Tween (PBST). 2 mL of the pooled and diluted patient sera was used as a primary antibody, and an HRP-tagged anti-human IgE mouse monoclonal antibody (Southern Biotech) was used as a secondary antibody. Separately, transferred proteins were stained using an HC121 antibody that recognizes both Jug r 1 and Jug r 2.

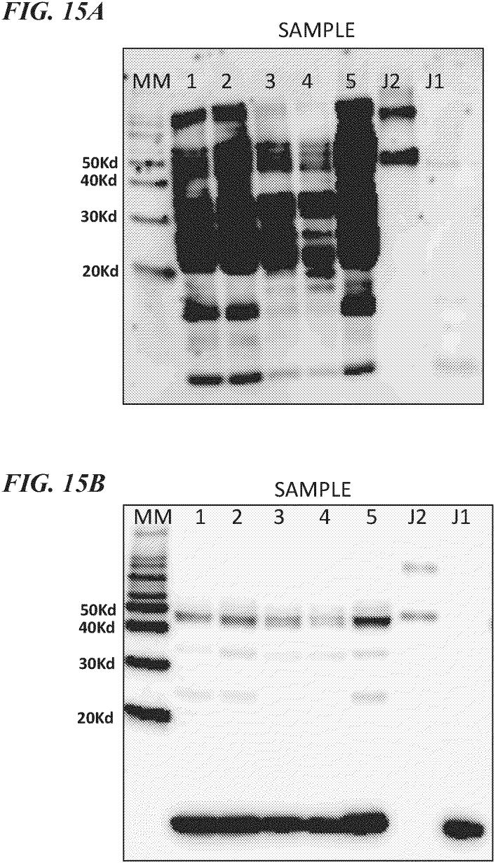

The immunoblot for the samples stained with the pooled patient sera is shown in FIG. 15A (“MM” indicates the molecular weight marker, “J1” indicates Jug r 1, and “J2” indicates Jug r 2.). Jug r 1 and Jug r 2 were detected in each of the five samples, although samples 1, 2 and 5 (each defatted using supercritical carbon dioxide) had higher Jug r 1 and Jug r 2 levels than samples 3 and 4.

The immunoblot for the samples stained with the HC121 antibody is shown in FIG. 15B. Similar to the immunoblot stained with the pooled patient sera, Jug r 1 and Jug r 2 were detected in each of the five samples. However, samples 1, 2 and 5 (each defatted using supercritical carbon dioxide) had higher Jug r 1 and Jug r 2 levels than samples 3 and 4.

### IX. Conclusion

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural or singular number, respectively. Additionally, the words “herein,” “above” and “below” and words of similar import, when used in this application, shall refer to this application as a whole and not to any particular portions of this application.

All publications, patents, and patent applications mentioned in this specification are herein incorporated by reference to the same extent as if each individual publication, patent, or patent application was specifically and individually indicated to be incorporated by reference.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while process steps, formulation components or functions are presented in a given order, alternative embodiments may include these in a different order, or substantially concurrently. The teachings of the disclosure provided herein can be applied to other compositions, not only the compositions described herein. The various embodiments described herein can be combined to provide further embodiments.

Specific elements of any of the foregoing embodiments can be combined or substituted for elements in other embodiments. Furthermore, while aspects associated with certain embodiments of the disclosure have been described in the context of these embodiments, other embodiments may also exhibit such aspects, and not all embodiments need necessarily exhibit such aspects to fall within the scope of the disclosure. Accordingly, the disclosure is not limited, except as by the appended claims.

What is claimed is:

1. A method of manufacturing an ultra-low fat nut material, the method comprising:
contacting a nut material having an initial oil content with a supercritical fluid to provide a defatted nut material having a reduced oil content; and
milling the defatted nut material.

2. A method of manufacturing an ultra-low fat nut material, the method comprising:
contacting a nut material having an initial oil content with a supercritical fluid to provide a defatted nut material having a reduced oil content; and
measuring a dose of the defatted nut material for oral immunotherapy.

3. The method of claim 2, wherein the measured dose of the defatted nut material comprises about 0.1 mg to about 1000 mg nut protein.

4. A method of manufacturing an ultra-low fat nut material, the method comprising:
contacting a nut material having an initial oil content with a supercritical fluid to provide a defatted nut material having a reduced oil content; and
packaging the defatted nut material in a package.

5. The method of claim 2, comprising combining the defatted nut material with a carrier material.

6. The method of claim 5, wherein the carrier material comprises one or more diluents, glidants, or lubricants.

7. The method of claim 2, wherein the defatted nut material has an oil content between 0.1% and 12%.

8. The method of claim 2, wherein the supercritical fluid is carbon dioxide.

9. The method of claim 2, wherein the nut material is a peanut material.

10. The method of claim 2, wherein the nut material is a tree nut material.

11. The method of claim 2, further comprising characterizing one or more nut protein allergens in the defatted nut material.

12. The method of claim 2, further comprising separating larger particles in the nut flour from smaller nut particles in the nut flour, and retaining the smaller nut particles.

13. The method of claim 2, wherein contacting the nut material with the supercritical fluid comprises flowing the supercritical fluid through the nut material.
